(19) United States
(12) Patent Application Publication
(10) Pub. No.: US 2025/0075213 A1
(43) Pub. Date: Mar. 6, 2025

(54) METHODS AND COMPOSITIONS FOR TREATING CARDIOMYOPATHY

(71) Applicant: ARIZONA BOARD OF REGENTS ON BEHALF OF THE UNIVERSITY OF ARIZONA, Tucson, AZ (US)

(72) Inventor: Christopher Pappas, Tucson, AZ (US)

(21) Appl. No.: 18/816,349

(22) Filed: Aug. 27, 2024

Related U.S. Application Data
(60) Provisional application No. 63/579,214, filed on Aug. 28, 2023.

(51) Int. Cl. C12N 15/113; C12Q 1/6883

(52) U.S. Cl. CPC C12N 15/113; C12Q 1/6883; C12Q 2600/156

(57) ABSTRACT

Nonsense-mediated mRNA decay, a cellular mechanism that eliminates mRNAs containing premature termination codons, underlies the loss of mutant protein caused by two independent disease-causing mutations (e.g., cardiomyopathy) in LMOD2. To counteract this, steric-blocking oligonucleotides (SBOs) were developed to obstruct the deposition of the exon junction complex, thereby preventing nonsense-mediated mRNA decay of the mutant LMOD2 transcripts and restoring mutant protein expression. This approach forms the basis for methods of treating subjects suffering from cardiomyopathy using these SBOs.

### CROSS-REFERENCE TO RELATED APPLICATIONS

This application is a non-provisional and claims benefit of U.S. Provisional Application No. 63/579,214 filed Aug. 28, 2023, the specification of which is incorporated herein in their entirety by reference.

### STATEMENT REGARDING FEDERALLY SPONSORED RESEARCH OR DEVELOPMENT

This invention was made with government support under Grant No. HL123078 awarded by National Institutes for Health. The government has certain rights in the invention.

### REFERENCE TO AN ELECTRONIC SEQUENCE LISTING

The contents of the electronic sequence listing (ARIZ 23_18 NP.xml; Size: 58,429 bytes; and Date of Creation: Aug. 21, 2024) is herein incorporated by reference in its entirety.

### FIELD OF THE INVENTION

The present invention features methods and compositions that allow for treating cardiomyopathy by treating a subject with steric-blocking oligonucleotides (SBOs) to restore mutant leiomodin 2 (LMOD2) protein expression.

### BACKGROUND OF THE INVENTION

The leiomodin (Lmod) family of actin-binding proteins is critical for the proper function of all types of muscle in humans. A biallelic mutation in LMOD1, the smooth muscle-specific LMOD family member, leads to megacystis microcolon intestinal hypoperistalsis syndrome, a rare visceral myopathy characterized by the defective contractile activity of muscles that line the bladder and intestine. Homozygous or compound heterozygous mutations in LMOD3, the isoform predominant in skeletal muscle, result in nemaline myopathy, a skeletal muscle disorder characterized by muscle weakness (there are >20 mutations reported to date). Additionally, mutations in the cardiac-predominant isoform LMOD2 have been linked to severe neonatal dilated cardiomyopathy (DCM). A second case confirmed the pathogenicity of this mutation. Additional mutations in LMOD2 have been discovered that also result in early-onset DCM and death. All data to date suggest that Lmod-linked muscle diseases have the common underlying pathophysiology of short, thin filaments, reduced muscle contractility, and severe muscle weakness. Most disease-causing mutations in the LMOD family of genes are nonsense or frameshift mutations, which would be predicted to result in the expression of truncated proteins. However, in most cases, little to no LMOD protein is expressed. In the case of the patient with the first described disease-causing mutation in LMOD2 (p.W398*), LMOD2 protein was not detected in the explanted heart via western blot analysis, and RT-qPCR analysis revealed a decrease in mature LMOD2 mRNA levels, but no change in pre-mRNA levels. Thus, nonsense-mediated mRNA decay (NMD) underlies the loss of mutant protein expression. Two additional mutations in LMOD2 that are associated with human disease (p.R513* and p.L415Vfs*108) are predicted to produce truncated proteins; however, information is lacking regarding whether and to what extent, protein is expressed.

NMD is a surveillance system whereby the cell detects and degrades mRNAs that contain premature termination codons (PTCs); however, NMD also regulates the levels of certain non-mutated, functional transcripts. Although not completely understood, there are multiple proposed pathways by which transcripts are identified and fated for degradation. One mechanism involves deposition of the multi-protein exon junction complex (EJC) upstream of exon-exon boundaries during RNA splicing. The EJC remains bound to the transcript and is normally removed by the ribosome during translation. However, if a PTC results in stalling of the ribosome, downstream EJCs can activate degradation of the transcript.

Thus, the present invention determined that multiple human LMOD2 mutations associated with dilated cardiomyopathy lead to a lack of LMOD2 protein expression through NMD and has developed steric-blocking oligonucleotides to specifically inhibit NMD of LMOD2 transcripts, increasing the levels of mutant-truncated protein. This treatment could potentially improve cardiac morphology and contractility for patients with certain mutations in LMOD2.

### BRIEF SUMMARY OF THE INVENTION

It is an objective of the present invention to provide methods and compositions that allow for treating cardiomyopathy by treating a subject with steric-blocking oligonucleotides (SBOs) to restore mutant leiomodin 2 (LMOD2) protein expression, as specified in the independent claims. Embodiments of the invention are given in the dependent claims. Embodiments of the present invention can be freely combined with each other if they are not mutually exclusive.

In some embodiments, the present invention features a method of treating a subject suffering from cardiomyopathy using a steric-blocking oligonucleotide (SBO). The method may comprise determining whether the subject carries a nonsense mutation in the LMOD2 gene. This determination may be made by obtaining or having obtained a biological sample from the subject and performing or having performed a genotyping assay (e.g., sequencing) on the biological sample to detect the presence of nonsense mutations in the LMOD2 gene. In some embodiments, if a nonsense mutation is identified, the method comprises administering an SBO to the subject. Alternatively, in some embodiments, if a nonsense mutation is identified, one or more SBOs are administered to the subject. In some embodiments, the biological samples may comprise whole blood; however, any biological sample containing intact cells, such as saliva or tissue, may also be utilized.

In other embodiments, the present invention may feature a method of treating cardiomyopathy in a subject in need thereof. The method may comprise administering a steric-blocking oligonucleotide (SBO) to a subject having cardiomyopathy, to prevent degradation of leiomodin 2 (LMOD2) messenger RNA (mRNA). Alternatively, in some embodiments, the method may comprise administering one or more steric-blocking oligonucleotides (SBOs) to a subject having cardiomyopathy, to prevent degradation of leiomodin 2 (LMOD2) messenger RNA (mRNA).

In some embodiments, the SBOs described herein inhibit nonsense-mediated decay (NMD) and thus prevent degradation of LMOD2 mRNA.

In further embodiments, the present invention features a steric-blocking oligonucleotide (SBO) comprising a sequence according to SEQ ID NO: 23, SEQ ID NO: 24, SEQ ID NO: 25, SEQ ID NO: 26; SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or SEQ ID NO: 41. In some embodiments, the SBO prevents degradation of a leiomodin 2 (LMOD2) mRNA.

One of the unique and inventive technical features of the present invention is the utilization of steric-blocking oligonucleotides (SBOs) to target mutant LMOD2. Without wishing to limit the invention to any theory or mechanism, it is believed that the technical feature of the present invention advantageously provides for a novel method to treat cardiomyopathy by preventing degradation of mutant leiomodin 2 (Lmod2) mRNA, thereby maintaining its protein expression. None of the presently known prior references or works have the unique inventive technical feature of the present invention.

Moreover, the prior references teach away from the present invention. For example, while general treatments for dilated cardiomyopathy—such as Beta-blockers, ACE inhibitors, and diuretics—are well-established, they are insufficient for patients with Lmod2 mutations. These patients often progress to a stage where such treatments lose their efficacy, necessitating circulatory support and, ultimately, cardiac transplantation. Notably, no existing therapies specifically target Lmod2.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, 1C, and 1D show human disease-causing mutations in LMOD2 result in a reduction in mutant protein levels when expressed from a construct containing a downstream intron. FIG. 1A shows a schematic of human LMOD2 constructs. Boxes represent exons, and intervening lines represent introns. The translational start and stop sites are indicated.

FIG. 1B-1D show western blot analysis of LMOD2 and GFP protein levels in lysate from AD-293 cells transfected with human LMOD2 gene constructs without (−) or with (+) the disease-causing mutations. FIG. 1B shows LMOD2 W398*. FIG. 1C shows LMOD2 R513*. FIG. 1D shows LMOD2 L415Vfs*108. To control for potential transfection differences LMOD2 protein levels were normalized to co-transfected GFP. Mutant protein levels are presented as a percentage of wild-type (WT) protein levels. Data are presented as means±SD. n=2-3; ****P<0.0001, One-way ANOVA with Tukey's multiple comparison test (FIG. 1B); n=3; *P<0.05, **P<0.01, Student's t-test (FIGS. 1C and 1D).

FIG. 2 shows a schematic of human LMOD2 protein with disease-causing mutations marked. LMOD2 contains a tropomyosin binding site (TMBS) and three actin-binding sites (ABS1,2,3). ABS2 is comprised of a leucine-rich repeat motif (LRR), and ABS3 is a WASP-homology 2, or Wiskott-Aldrich homology 2, (WH2) domain. Red bar (indicated with a star) denotes the length of random amino acids that follow the L415V frameshift. p., protein.

FIGS. 3A and 3B show human disease-causing mutations in LMOD2 result in a decrease in mature, but not pre-, mRNA levels when expressed from a construct containing a downstream intron, which is ameliorated by blocking translation. FIG. 3A shows RT-qPCR analysis of LMOD2 mature mRNA and pre-mRNA levels in AD-293 cells expressing LMOD2 coding sequence (CDS) or minigene constructs (MG) containing the W398* (left panel) or R513* (right panel) mutations, shown as a percentage of wild type (WT) mRNA levels. FIG. 3B shows RT-qPCR analysis of LMOD2 mature mRNA and pre-mRNA levels in AD-293 cells expressing LMOD2 minigene (MG) constructs containing the W398* (left panel) or R513* (right panel) mutations treated with vehicle alone (VEH) or 100 M cycloheximide (CHX) for 4-5 h, shown as a percentage of wild type (WT) mRNA levels. Data are presented as means±SD; n=3-5; *P<0.05, **P<0.01, Student's t-test.

FIGS. 4A, 4B, and 4C show nonsense-mediated mRNA decay is comparable in cells stably expressing of LMOD2 [W398*] minigene constructs. LMOD2 [W398*] mutant protein is slightly less stable than WT protein. FIG. 4A shows a western blot analysis of LMOD2 protein levels in FT-293 cells stably expressing LMOD2 gene constructs without (−) or with (+) the W398* mutation, presented as a percentage of wild-type (WT) protein levels. FIG. 4B shows RT-qPCR analysis of LMOD2 mature mRNA (left) and pre-mRNA (right) levels in FT-293 cells stably expressing LMOD2 coding sequence (CDS) or minigene constructs (MG) containing the W398* mutation, shown as a percentage of wild type (WT) mRNA levels. FIG. 4C shows a cycloheximide (CHX) chase experiment. LMOD2 protein levels in FT-293 cells stably expressing the LMOD2 CDS construct without or with the W398* mutation treated with 300 M CHX for 4, 8, or 24 h. Data are presented as means±SD; n=2-8 (FIG. 4A), 3 (FIG. 4B), and 4 (FIG. 4C); *P<0.05, **P<0.01, ****P<0.0001, Student's t-test.

FIGS. 5A, 5B, 50, 5D, 5E, 5F, and 5G show nonsense-mediated mRNA decay of the LMOD2 [W398*] minigene construct is inhibited by treatment with steric-blocking oligonucleotides that hinders deposition of the EJC. FIG. 5A shows a normalized firefly luciferase activity for Luc-LMOD2 CDS and MG constructs with and without the W398* (left) or R513* (right) mutations presented as a percentage of wild-type (WT) protein levels. Data are presented as means±SD. n=4; ****P<0.0001, Student's t-test. FIG. 5B shows a schematic of the LMOD2-MG construct with boxes representing exons and intervening lines representing introns. The locations of the W398* mutation, SBOs, and the putative EJC binding site (between −24 and −20) upstream of the junction between exons 2 and 3 are indicated. FIG. 5C shows normalized firefly luciferase activity for Luc-LMOD2 MG [W398*] treated with 100 nM of the steric-blocking oligonucleotides (SBOs) indicated. Data are presented as the mean percentage of Luc-LMOD2 MG (WT)+Scr SBO activity±SD. n=3; *P<0.05; **P<0.01; ***P<0.001, One-way ANOVA with Dunnett's multiple comparison test. FIG. 5D shows the normalized firefly luciferase activity for Luc-LMOD2 MG [W398*] treated with various concentrations of the L2 [−38] SBO. Data are presented as the mean percentage of Luc-LMOD2 MG+100 nM scrambled SBO±SD. n=4; ***P<0.001, ****P<0.0001, One-way ANOVA with Dunnett's multiple comparison test. FIG. 5E shows the normalized firefly luciferase activity for Luc-LMOD2 MG [R513*] treated with 400 nM of the steric-blocking oligonucleotides (SBOs) indicated. Data are presented as the mean percentage of Luc-LMOD2 MG (WT) activity±SD. n=3; **P<0.01, Student's t-test. FIG. 5F shows a representative western blot of input or immunoprecipitate probed for components of the EJC (EIF4A3 and MAGOH) or control proteins (Vinculin and GAPDH). FIG. 5G shows RT-qPCR analysis of LMOD2 mature mRNA levels in input lysate (left panel) or the immunoprecipitate (right panel) from cells treated with 100 nM scrambled (Scr) or L2 [−38] SBOs. Data are presented as means±SD. n=3; *P<0.05, **P<0.01, comparison with a hypothetical mean of 1.0, one sample t-test.

FIGS. 6A and 6B show the treatment with the steric-blocking oligonucleotides L2 [−30], L2 [−34] and L2 [−38] recovers LMOD2 [W398*] mutant protein and mature mRNA levels. FIG. 6A shows the western blot analysis of LMOD2 protein levels in FT-293 cells stably expressing LMOD2 minigene (MG) constructs with or without the W398* mutation and treated with the indicated SBOs. Relative expression levels were determined following normalization to total protein levels assessed via Ponceau S staining. Bars represent negative control (−), scramble (Scr), L2 [−26], L2 [−30], L2 [−34], and L2 [−38] from left to right. Data are presented as means±SD. n=4; ***P<0.001, ****P<0.0001, Two-way ANOVA with Dunnett's multiple comparison test. FIG. 6B shows an RT-qPCR analysis of LMOD2 mature mRNA (left) and pre-mRNA (right) levels in FT-293 cells stably expressing LMOD2 minigene constructs (MG) containing the W398* mutation treated with the indicated SBOs. Data are presented as means±SD. n=9; *P<0.05, **P<0.01, ****P<0.0001, One-way ANOVA with Dunnett's multiple comparison test.

### DETAILED DESCRIPTION OF THE INVENTION

Disclosed are various peptides, solvents, solutions, carriers, and/or components to be used to prepare compositions to be used within the methods disclosed herein. Also disclosed are the various steps, elements, amounts, routes of administration, symptoms, and/or treatments that are used or observed when performing the disclosed methods, as well as the methods themselves. These and other materials, steps, and/or elements are disclosed herein, and it is understood that when combinations, subsets, interactions, groups, etc. of these materials are disclosed, that while specific reference of each various individual and collective combination and permutation of these compounds may not be explicitly disclosed, each is specifically contemplated and described herein. It is also to be understood that the terminology used herein is for the purpose of describing particular embodiments only and is not intended to be limiting.

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising,” such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder or condition described herein. In another embodiment, the subject is a mammal (e.g., a human) at risk of developing a disease, disorder, or condition described herein. In certain instances, the term patient refers to a human.

As used herein, the terms “treat,” “treating,” or “treatment” refer to both therapeutic treatment and prophylactic or preventative measures, with the objective of preventing, reducing, slowing down (lessen), inhibiting, or eliminating an undesired physiological change, symptom, disease, or disorder. For example, the disease may be dilated cardiomyopathy (DCM). For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the subject or patient may be identified (e.g., diagnosed) as one suffering from the disease or condition prior to administration of the compositions of the invention. Subjects at risk for the disease can be identified by, for example, any or a combination of appropriate diagnostic or prognostic assays known in the art.

As used herein, “clinical improvement” may refer to a noticeable reduction in the symptoms of a disorder, or cessation thereof.

The terms “manage,” “managing,” and “management” refer to preventing or slowing the progression, spread, or worsening of a disease or disorder or of one or more symptoms thereof. In certain cases, the beneficial effects that a subject derives from a prophylactic or therapeutic agent do not result in a cure of the disease or disorder.

The terms “regress,” “regressing,” and “regression” may refer to a decrease in the size of a tumor or in the extent of cancer in the body. In some embodiments, “regression” may refer to a decrease in severity of the disease and/or decrease in the size of a tumor. In some embodiments, regression may generally refer to lighter symptoms without the disease completely disappearing. In certain cases, the beneficial effects that a subject derives from a prophylactic or therapeutic agent do not result in a cure of the disease or disorder. In some embodiments, symptoms of the disease may return.

The terms “administering” and “administration” refer to methods of providing a pharmaceutical preparation to a subject. Such methods are well known to those skilled in the art and include, but are not limited to, administering the compositions orally, parenterally (e.g., intravenously and subcutaneously), by intramuscular injection, by intraperitoneal injection, intrathecally, transdermally, extracorporeally, intranasally, topically, or the like.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained.

A “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors, including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single-dose compositions can contain such amounts or submultiples thereof to make up the daily dose. The dosage can be adjusted by the individual physician in the event of any contraindications. Dosage can vary, and can be administered in one or more dose administrations daily, for one or several days, weekly, twice weekly, etc. Guidance can be found in the literature for appropriate dosages for given classes of pharmaceutical products.

The exact amount of the compositions required will vary from subject to subject, depending on the species, age, weight, and general condition of the subject, the severity of the disorder being treated, the particular composition used, its mode of administration, and the like. Thus, it is not possible to specify an exact amount for every composition. However, an appropriate amount can be determined by one of ordinary skill in the art using only routine experimentation given the teachings herein.

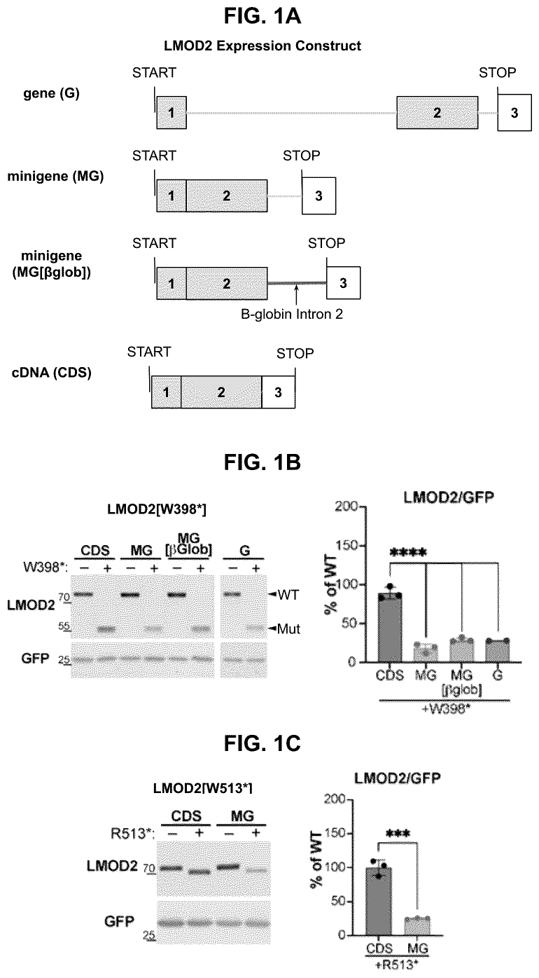

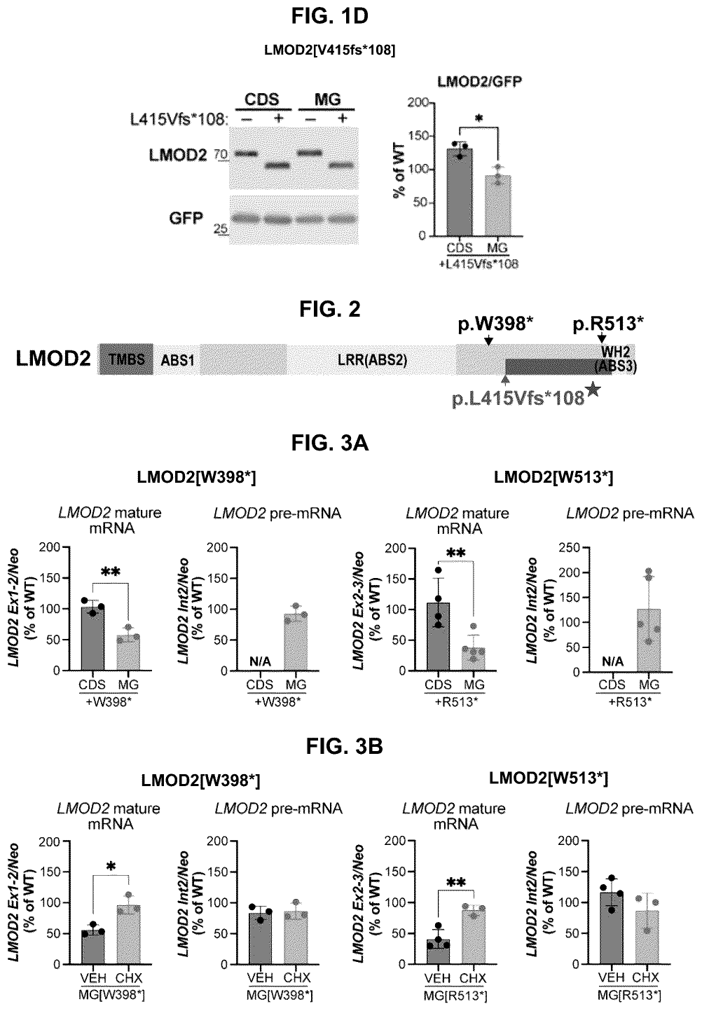

Referring now to FIGS. 1A-6B, the present invention features methods and compositions designed to treat cardiomyopathy. Specifically, the present invention utilizes steric-blocking oligonucleotides (SBOs) to restore the expression of mutant leiomodin 2 (LMOD2) protein in affected subjects

The present invention features methods for treating cardiomyopathy in a subject in need thereof. In some embodiments, the method comprises identifying a subject presenting with cardiomyopathy; and administering a steric-blocking oligonucleotide (SBO) to the subject to prevent degradation of mutant leiomodin 2 (LMOD2) mRNA, thereby maintaining its protein expression. In other embodiments, the method comprises identifying a subject presenting with cardiomyopathy; and administering one or more steric-blocking oligonucleotide (SBO) to the subject to prevent degradation of mutant leiomodin 2 (LMOD2) mRNA, thereby maintaining its protein expression.

Cardiomyopathy is typically diagnosed initially through the observation of clinical symptoms, such as desaturations, which are subsequently confirmed by cardiac imaging techniques, such as echocardiography. In certain embodiments of the present invention, identifying a subject presenting with cardiomyopathy involves determining the presence of nonsense mutations in the LMOD2 gene through DNA sequencing, thereby pinpointing individuals who are suitable candidates for SBO treatment.

Additionally, the present invention may include methods for treating a subject suffering from cardiomyopathy using a steric-blocking oligonucleotide (SBO). In some embodiments, the method comprises determining whether the subject carries a nonsense mutation in the LMOD2 gene. This determination may be made by: (a) obtaining or having obtained a biological sample from the subject; (b) performing or having performed a genotyping assay (e.g., sequencing) on the biological sample to detect the presence of nonsense mutations in the LMOD2 gene; and, if a nonsense mutation is identified, administering an SBO to the subject. Alternatively, in some embodiments, if a nonsense mutation is identified, one or more SBOs are administered to the subject. In some embodiments, the biological samples may comprise whole blood; however, any biological sample containing intact cells, such as saliva or tissue, may also be utilized.

Additionally, the methods may include administering a stop-readthrough drug in combination with SBOs to facilitate the production of non-mutated, full-length LMOD2 protein. Non-limiting examples of stop-readthrough drugs may include aminoglycosides such as gentamicin or the oxadiazole compound Ataluren.

In some embodiments, the methods and compositions disclosed herein are used to treat dilated cardiomyopathy resulting from nonsense mutations in the LMOD2 gene.

In certain embodiments, the LMOD2 gene may contain a point mutation, e.g., a nonsense mutation, which introduces a stop codon, causing premature termination of the protein sequence. For instance, nonsense mutations may occur at specific amino acids, such as W398 or R513, or at both locations. Without intending to limit the scope of the present invention, it is believed that the aforementioned mutations activate nonsense-mediated mRNA decay, which in turn leads to cardiomyopathy.

In some embodiments, the steric-blocking oligonucleotides (SBOs) are administered via an intravenous injection. In other embodiments, the SBOs are administered via a subcutaneous injection.

In some embodiments, the SBO inhibits nonsense-mediated mRNA decay (NMD) by blocking deposition of the exon junction complex. In some embodiments, the SBO is uniformly modified with 2′-O-methoxyethyl (MOE) nucleotides and phosphorothioate linkages. In some embodiments, all nucleotides in the SBOs are modified with 2′-O-methoxyethyl (MOE) groups and phosphorothioate linkages. In other embodiments, all nucleotides in the SBOs are modified with 2′-O-methoxyethyl (MOE) groups, with at least one phosphorothioate linkage included.

Without wishing to limit the present invention to any theory or mechanism, it is believed that the 2′-O-methoxyethyl (MOE) modification provides protection against nucleases, prevents cleavage by RNase H and increases the SBOs affinity for the target RNA. While not all steric-blocking oligonucleotides (SBOs) may require phosphorothioate linkages—since the 2′-MOE modification itself imparts nuclease resistance—the inclusion of phosphorothioate linkages may enhance protein binding, thereby improving delivery and cellular uptake. Consequently, the pharmacokinetics of SBOs can be optimized by adjusting the number of phosphorothioate linkages.

In some embodiments, the SBO is selected from one or a combination of SEQ ID NO: 23, SEQ ID NO: 24, SEQ ID NO: 25, SEQ ID NO: 26; SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 41. The present invention is not limited to the aforementioned sequences and may encompass other sequences of any length that inhibit nonsense-mediated decay of mutant LMOD2 mRNA, thereby preserving its protein expression.

In some embodiments, the SBOs comprise sequences that are at least 95% identical to the aforementioned sequences (e.g., SEQ ID NO: 23-SEQ ID NO: 41). In some embodiments, the SBOs comprise sequences that are at least 90% identical to the aforementioned sequences. In some embodiments, the SBOs comprise sequences that are at least 85% identical to the aforementioned sequences. In some embodiments, the SBOs comprise sequences that are at least 80% identical to the aforementioned sequences.

In certain embodiments, the SBO is one or more of the following: SEQ ID NO: 38 (L2 [−38]), SEQ ID NO: 34 (L2 [−34]), or SEQ ID NO: 30 (L2 [−30]).

The present invention may also feature a steric-blocking oligonucleotide (SBO) comprising a sequence according to SEQ ID NO: 23, SEQ ID NO: 24, SEQ ID NO: 25, SEQ ID NO: 26; SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or SEQ ID NO: 41. In some embodiments, the SBO prevents degradation of a leiomodin 2 (LMOD2) mRNA. In some embodiments, the present invention features a steric-blocking oligonucleotide (SBO) comprising at least one of the following: L2 [−38], L2 [−34], and L2 [−30]. In certain embodiments, the LMOD2 gene may contain a nonsense mutation. For instance, nonsense mutations may occur at specific amino acids, such as W398 or R513, or both locations. In some embodiments, the SBOs bind LMOD2 mRNA preventing deposition of the exon junction complex thereby disrupting activation of nonsense-mediated mRNA decay (NMD).

### Example

The following is a non-limiting example of the present invention. It is to be understood that said example is not intended to limit the present invention in any way. Equivalents or substitutes are within the scope of the present invention.

Human embryonic kidney 293 (HEK293) cell lines/Transfections: AD-293 cells (Agilent, 240085), a derivative of HEK293 cells, were cultured in DMEM (Gibco, 11995-065) supplemented with 8% fetal bovine serum (Omega Scientific, FB-02), MEM non-essential amino acids (Gibco, 11140-050) and Pen-Strep (Gibco, 15140-122). Cells were transfected using Lipofectamine 3000 Transfection Reagent with a lipid-to-DNA ratio of 2:1 according to the manufacturer's instructions (Invitrogen, L3000015). Cells were collected 48-72 hours after transfection. To generate isogenic stable HEK293 cell lines expressing LMOD2 gene constructs, the Flp-In™ system was used according to the manufacturer's instructions (ThermoFisher Scientific, K601002). Briefly, a host TREx-293 cell line was purchased, which contains a Flp Recombination Target (FRT) site at a single locus in the genome (ThermoFisher Scientific, R71007). Co-transfection of an expression vector (pcDNA5/FT, that contains an LMOD2 gene construct under control of a CMV promoter, an FRT site, and a hygromycin resistance gene) with a plasmid expressing Flp recombinase (pOG44) results in homologous recombination between the Flp sites and integration of the LMOD2 construct into a single site within the genome. “Polyclonal” selection of stably expressing cell lines (named Flp-In™ TREx-293 [FT-293]) was then completed via hygromycin selection. The expression plasmid used (pcDNA5/FT) results in constitutive LMOD2 expression.

Cycloheximide (CHX) treatment: For experiments to inhibit NMD, AD-293 cells were plated at 700,000 cells/35 mm culture dish, transfected the next day (at 50-80% confluency) with LMOD2 gene constructs, and treated after 2 days of expression with 100 g/mL of CHX resuspended in EtOH for 4-5 h (Sigma, C7698). For CHX chase experiments, FT-293 cells were plated at 1×10⁶ cells/35 mm culture dish, and after 2 days treated with 300 M CHX in DMSO (Sigma, 508739) for 0, 4, 8, and 24 h.

Lmod2 gene constructs: LMOD2 and Hemoglobin subunit beta (HBB) gene sequences (corresponding to the NCBI human reference sequences GRCh38.14; chr7:g.123655866-g.123664290 and chr11: complement, g.522546-g.5227071, respectively) were cloned from human genomic DNA and the LMOD2 coding sequence synthesized according to NCBI reference sequence NM_207163.2 (Genscript). To generate minigene (MG) constructs, fragments subcloned from the gene and cDNA sequences were combined by overlap extension PCR. Gene, minigene, and cDNA sequences were inserted into pcDNA3.1 (−) or pcDNA5/FRT, placing them under the control of a CMV promoter. Note the constructs do not contain the 5′ UTR of LMOD2. LMOD2 disease-causing mutations were added to the constructs via site-directed mutagenesis as previously described. Relevant sequence information is in Table 1 and Table 2.

TABLE 1

| Site-directed Mutagenesis | | SEQ |
|---|---|---|
| Primers | Sequences | ID NO: |
| hLMOD2 c.1193G > A, | CTTATGTATCTCCCAGGCACTCACCCTAGTCAT | 1 |
| p.W398* (Forward) | CC | |
| hLMOD2 c.1193G > A, | CCTGGGAGATACATAAGGTGAAGAGCTAGGTG | 2 |
| p.W398* (Reverse) | TTCCT | |
| hLMOD2 c.1243_1244 del, | TCCAGACTGTGAGGAGCCGTCCTGTCTCCTGT | 3 |
| p.L415Vfs*108 (Forward) | GG | |
| hLMOD2 c.1243_1244 del, | CTCCTCACAGTCTGGACTTTTTTGGGGAGTTTT | 4 |
| p.L415Vfs*108 (Reverse) | GGGGATG | |
| hLMOD2 c.1537C > T, p.R513* | CAGTTCCTGACCTTCTACCCCACAGAGATCAG | 5 |
| (Forward) | CTC | |
| hLMOD2 c.1537C > T, p.R513* | GAAGGTCAGGAACTGTCTTCCATTTTCTTCTCT | 6 |
| (Reverse) | TGCAC | |
| | | SEQ |
| RT-qPCR Primers | Sequences | ID NO: |
| LMOD2 Ex1-2 (spans intron 1) | GGACATTCAGCAGAGAGGCACT | 7 |
| (Forward) | | |
| LMOD2 Ex1-2 (spans intron 1) | GATAAGCTCTTCTTCACTTTCCTC | 8 |
| (Reverse) | | |

128-bp product; E = 1.83

| | | |
|---|---|---|
| LMOD2 Ex2-3 (spans intron 2) | ACCCCACAGAGATCAGCTCA | 9 |
| (Forward) | | |
| LMOD2 Ex2-3 (spans intron 2) | TATCGCAGGGCTTCTGGAAC | 10 |
| (Reverse) | | |

98-bp product; E = 1.909

| | | |
|---|---|---|
| LMOD2 Int2 (located within | ACATTAGGAGCAGGCACACA | 11 |
| intron 2) (Forward) | | |
| LMOD2 Int2 (located within | AGGGAATTGGGACGTGTATGA | 12 |
| intron 2) (Reverse) | | |

119-bp product; E = 1.979

| | | |
|---|---|---|
| RPL32 (Forward) | CACCAGTCAGACCGATATGTCAAAA | 13 |
| RPL32 (Reverse) | TGTTGTCAATGCCTCTGGGTTT | 14 |

64-bp product; E = 1.912

| | | |
|---|---|---|
| Neomycin resistance gene | GATGGATTGCACGCAGGTTC | 15 |
| (Forward) | | |
| Neomycin resistance gene | TCAGAGCAGCCGATTGTCTG | 16 |
| (Reverse) | | |

86-bp product; E = 1.787

| | | |
|---|---|---|
| Hygromycin resistance gene | ACAGCGGTCATTGACTGGAG | 17 |
| (Forward) | | |
| Hygromycin resistance gene | TGCTGCTCCATACAAGCCAA | 18 |
| (Reverse) | | |

101-bp product; E = 1.827
Note:
E stands for amplification efficiency of the primer set

TABLE 2

| | | SEQ ID |
|---|---|---|
| | Sequences | NO: |
| LMOD2 | ATGTCTACCTTTGGCTACCGAAGAGGACTCAGTAAATACGAAT | 19 |
| cDNA (CDS) | CCATCGACGAGGATGAACTCCTCGCCTCCCTGTCAGCCGAGG | |
| | AGCTGAAGGAGCTAGAGAGAGAGTTGGAAGACATTGAACCTGA | |
| | CCGCAACCTTCCCGTGGGGCTAAGGCAAAAGAGCCTGACAGA | |
| | GAAAACCCCCACAGGGACATTCAGCAGAGAGGCACTGATGGC | |
| | CTATTGGGAAAAGGAGTCCCAAAAACTCTTGGAGAAGGAGAGG | |
| | CTGGGGGAATGTGGAAAGGTTGCAGAAGACAAAGAGGAAAGT | |
| | GAAGAAGAGCTTATCTTTACTGAAAGTAACAGTGAGGTTTCTGA | |
| | GGAAGTGTATACAGAGGAGGAGGAGGAGGAGTCCCAGGAGGA | |
| | AGAGGAGGAAGAAGACAGTGACGAAGAGGAAAGAACAATTGA | |
| | AACTGCAAAAGGGATTAATGGAACTGTAAATTATGATAGTGTCA | |
| | ATTCTGACAACTCTAAGCCAAAGATATTTAAAAGTCAAATAGAG | |
| | AACATAAATTTGACCAATGGCAGCAATGGGAGGAACACAGAGT | |
| | CCCCAGCTGCCATTCACCCTTGTGGAAATCCTACAGTGATTGA | |
| | GGACGCTTTGGACAAGATTAAAAGCAATGACCCTGACACCACA | |
| | GAAGTCAATTTGAACAACATTGAGAACATCACAACACAGACCCT | |
| | TACCCGCTTTGCTGAAGCCCTCAAGGACAACACTGTGGTGAAG | |
| | ACGTTCAGTCTGGCCAACACGCATGCCGACGACAGTGCAGCC | |
| | ATGGCCATTGCAGAGATGCTCAAAGTCAATGAGCACATCACCA | |
| | ACGTAAACGTCGAGTCCAACTTCATAACGGGAAAGGGGATCCT | |
| | GGCCATCATGAGAGCTCTCCAGCACAACACGGTGCTCACGGA | |
| | GCTGCGTTTCCATAACCAGAGGCACATCATGGGCAGCCAGGT | |
| | GGAAATGGAGATTGTCAAGCTGCTGAAGGAGAACACGACGCT | |
| | GCTGAGGCTGGGATACCATTTTGAACTCCCAGGACCAAGAATG | |
| | AGCATGACGAGCATTTTGACAAGAAATATGGATAAACAGAGGC | |
| | AAAAACGTTTGCAGGAGCAAAAACAGCAGGAGGGATACGATG | |
| | GAGGACCCAATCTTAGGACCAAAGTCTGGCAAAGAGGAACACC | |
| | TAGCTCTTCACCTTATGTATCTCCCAGGCACTCACCCTGGTCAT | |
| | CCCCAAAACTCCCCAAAAAAGTCCAGACTGTGAGGAGCCGTCC | |
| | TCTGTCTCCTGTGGCCACACCTCCTCCTCCTCCCCCTCCTCCT | |
| | CCTCCTCCCCCTCCTTCTTCCCAAAGGCTGCCACCACCTCCTC | |
| | CTCCTCCCCCTCCTCCACTCCCAGAGAAAAAGCTCATTACCAG | |
| | AAACATTGCAGAAGTCATCAAACAACAGGAGAGTGCCCAACGG | |
| | GCATTACAAAATGGACAAAAAAAGAAAAAAGGGAAAAAGGTCA | |
| | AGAAACAGCCAAACAGTATTCTAAAGGAAATAAAAAATTCTCTG | |
| | AGGTCAGTGCAAGAGAAGAAAATGGAAGACAGTTCCCGACCTT | |
| | CTACCCCACAGAGATCAGCTCATGAGAATCTCATGGAAGCAAT | |
| | TCGGGGAAGCAGCATAAAACAGCTAAAGCGGGTGGAAGTTCC | |
| | AGAAGCCCTGCGATAA | |
| LMOD2 | ATGTCTACCTTTGGCTACCGAAGAGGACTCAGTAAATACGAAT | 20 |
| minigene | CCATCGACGAGGATGAACTCCTCGCCTCCCTGTCAGCCGAGG | |
| (MG) | AGCTGAAGGAGCTAGAGAGAGAGTTGGAAGACATTGAACCTGA | |
| | CCGCAACCTTCCCGTGGGGCTAAGGCAAAAGAGCCTGACAGA | |
| | GAAAACCCCCACAGGGACATTCAGCAGAGAGGCACTGATGGC | |
| | CTATTGGGAAAAGGAGTCCCAAAAACTCTTGGAGAAGGAGAGG | |
| | CTGGGGGAATGTGGAAAGGTTGCAGAAGACAAAGAGGAAAGT | |
| | GAAGAAGAGCTTATCTTTACTGAAAGTAACAGTGAGGTTTCTGA | |
| | GGAAGTGTATACAGAGGAGGAGGAGGAGGAGTCCCAGGAGGA | |
| | AGAGGAGGAAGAAGACAGTGACGAAGAGGAAAGAACAATTGA | |
| | AACTGCAAAAGGGATTAATGGAACTGTAAATTATGATAGTGTCA | |
| | ATTCTGACAACTCTAAGCCAAAGATATTTAAAAGTCAAATAGAG | |
| | AACATAAATTTGACCAATGGCAGCAATGGGAGGAACACAGAGT | |
| | CCCCAGCTGCCATTCACCCTTGTGGAAATCCTACAGTGATTGA | |
| | GGACGCTTTGGACAAGATTAAAAGCAATGACCCTGACACCACA | |
| | GAAGTCAATTTGAACAACATTGAGAACATCACAACACAGACCCT | |
| | TACCCGCTTTGCTGAAGCCCTCAAGGACAACACTGTGGTGAAG | |
| | ACGTTCAGTCTGGCCAACACGCATGCCGACGACAGTGCAGCC | |
| | ATGGCCATTGCAGAGATGCTCAAAGTCAATGAGCACATCACCA | |
| | ACGTAAACGTCGAGTCCAACTTCATAACGGGAAAGGGGATCCT | |
| | GGCCATCATGAGAGCTCTCCAGCACAACACGGTGCTCACGGA | |
| | GCTGCGTTTCCATAACCAGAGGCACATCATGGGCAGCCAGGT | |
| | GGAAATGGAGATTGTCAAGCTGCTGAAGGAGAACACGACGCT | |
| | GCTGAGGCTGGGATACCATTTTGAACTCCCAGGACCAAGAATG | |
| | AGCATGACGAGCATTTTGACAAGAAATATGGATAAACAGAGGC | |
| | AAAAACGTTTGCAGGAGCAAAAACAGCAGGAGGGATACGATG | |
| | GAGGACCCAATCTTAGGACCAAAGTCTGGCAAAGAGGAACACC | |
| | TAGCTCTTCACCTTATGTATCTCCCAGGCACTCACCCTGGTCAT | |
| | CCCCAAAACTCCCCAAAAAAGTCCAGACTGTGAGGAGCCGTCC | |
| | TCTGTCTCCTGTGGCCACACCTCCTCCTCCTCCCCCTCCTCCT | |
| | CCTCCTCCCCCTCCTTCTTCCCAAAGGCTGCCACCACCTCCTC | |
| | CTCCTCCCCCTCCTCCACTCCCAGAGAAAAAGCTCATTACCAG | |
| | AAACATTGCAGAAGTCATCAAACAACAGGAGAGTGCCCAACGG | |
| | GCATTACAAAATGGACAAAAAAAGAAAAAAGGGAAAAAGGTCA | |
| | AGAAACAGCCAAACAGTATTCTAAAGGAAATAAAAAATTCTCTG | |
| | AGGTCAGTGCAAGAGAAGAAAATGGAAGACAGTTCCCGACCTT | |
| | CTACCCCACAGAGATCAGCTCATGAGAATCTCATGGAAGCAAT | |
| | TCGGGGAAGCAGCATAAAACAGCTAAAGCGGGTAAGTAACCA | |
| | GAGAACAGACATAGGGGCACAGATAAAGTAAATGAGTTGTCCT | |
| | CCATTGCATGGTGGTACCAAAGTCACCTCTCACAATACTTATCA | |
| | ATACTTTCAATATTTTAGTATGCGAGAGCAAACACACCAAGTTT | |
| | GAAACATTAGGAGCAGGCACACAAGTGAGCACATTTCTATTTG | |
| | AGAGGAACGCCTGGGCCGCTTTCCCAGCCTCTCAATCATATAA | |
| | GGGCAAGGACCATTTTCATACACGTCCCAATTCCCTTAAGAAG | |
| | AAAACCAGGGAATATAATCTAAATTCCAAAAGGATTCACCACTT | |
| | GTCAAAAATTTTACCACAGAGTTGTGACTGATTCGATACCTAAT | |
| | TTATAACATAAAATTTATAATGTGGTAAAAAAATTACCATGCAGC | |
| | AATAATCAACCTGAGTTCTTCTCAGAGGATACTGCTAGAGACAT | |
| | ATCCTACAGATATTTTTCACTTATCATGTGTGTTGTTTCATTGAG | |
| | AGAAAATCCGCTATTTTTGTAGGTGGAAGTTCCAGAAGCCCTG | |
| | CGATAAAAACATGATCTTTAGAAGAGGATGCAGAACTGTTCAGT | |
| | GGTATTACATGAAATGCATTGTGAGATGTTTCTAAAATACCTTC | |
| | TTCAATTCAAAATGATCCCTGACTTTAAAAATAATCTCACCCATT | |
| | AATTCCAAAGAGAATCTTAAGAAACAATCAGCATGTTTCTTCTG | |
| | TAAATATGAAAATAAATTTCTTTTTTATGTCGTGAGATTTGTATT | |
| | GGCAAGAAGCAGTTAATTTAAAGATGCTCTTCCTATCTGTGGAT | |
| | GTGTTGGTAACTCCGAGTTGTAATGAGTTCATGAAATGTGCTGT | |
| | TATTTTTGTAATCTCAATAAATGTGGATTGAAGTTTTTTCCCTTT | |
| | TTTTAAAGCCAAACTAATATTTTTCTGTGACTTGATACATCTGTC | |
| | AGATTTTTGTAATCTCGATAAATGTGTATTGAAGTTTTTTCCCTT | |
| | TTTTTAAAAAGCCAAACTAATATTTTTCTGTGAGTTAATACATCT | |
| | GTCAGGTGTGTATGTAACATTACTGGACATTAAAAAAAAATATT | |
| | ACATTCTCACCCAAAAAGGGTTTGGGTCCTAAGCATTTGCCTTT | |
| | CTTTGTTTCCTCTTGTTCAAGAAAATCTGATTAGATCTCTTTCTA | |
| | AAGGACTGCAAGCAATAATTTTTTTTTATATTTTATTTATTTATTT | |
| | ATTTTTTTGAGACAAGTCTTGCTCTGTTACCCA | |
| LMOD2 | ATGTCTACCTTTGGCTACCGAAGAGGACTCAGTAAATACGAAT | 21 |
| minigene | CCATCGACGAGGATGAACTCCTCGCCTCCCTGTCAGCCGAGG | |
| (MG) + Beta- | AGCTGAAGGAGCTAGAGAGAGAGTTGGAAGACATTGAACCTGA | |
| globin intron | CCGCAACCTTCCCGTGGGGCTAAGGCAAAAGAGCCTGACAGA | |
| 2 | GAAAACCCCCACAGGGACATTCAGCAGAGAGGCACTGATGGC | |
| | CTATTGGGAAAAGGAGTCCCAAAAACTCTTGGAGAAGGAGAGG | |
| | CTGGGGGAATGTGGAAAGGTTGCAGAAGACAAAGAGGAAAGT | |
| | GAAGAAGAGCTTATCTTTACTGAAAGTAACAGTGAGGTTTCTGA | |
| | GGAAGTGTATACAGAGGAGGAGGAGGAGGAGTCCCAGGAGGA | |
| | AGAGGAGGAAGAAGACAGTGACGAAGAGGAAAGAACAATTGA | |
| | AACTGCAAAAGGGATTAATGGAACTGTAAATTATGATAGTGTCA | |
| | ATTCTGACAACTCTAAGCCAAAGATATTTAAAAGTCAAATAGAG | |
| | AACATAAATTTGACCAATGGCAGCAATGGGAGGAACACAGAGT | |
| | CCCCAGCTGCCATTCACCCTTGTGGAAATCCTACAGTGATTGA | |
| | GGACGCTTTGGACAAGATTAAAAGCAATGACCCTGACACCACA | |
| | GAAGTCAATTTGAACAACATTGAGAACATCACAACACAGACCCT | |
| | TACCCGCTTTGCTGAAGCCCTCAAGGACAACACTGTGGTGAAG | |
| | ACGTTCAGTCTGGCCAACACGCATGCCGACGACAGTGCAGCC | |
| | ATGGCCATTGCAGAGATGCTCAAAGTCAATGAGCACATCACCA | |
| | ACGTAAACGTCGAGTCCAACTTCATAACGGGAAAGGGGATCCT | |
| | GGCCATCATGAGAGCTCTCCAGCACAACACGGTGCTCACGGA | |
| | GCTGCGTTTCCATAACCAGAGGCACATCATGGGCAGCCAGGT | |
| | GGAAATGGAGATTGTCAAGCTGCTGAAGGAGAACACGACGCT | |
| | GCTGAGGCTGGGATACCATTTTGAACTCCCAGGACCAAGAATG | |
| | AGCATGACGAGCATTTTGACAAGAAATATGGATAAACAGAGGC | |
| | AAAAACGTTTGCAGGAGCAAAAACAGCAGGAGGGATACGATG | |
| | GAGGACCCAATCTTAGGACCAAAGTCTGGCAAAGAGGAACACC | |
| | TAGCTCTTCACCTTATGTATCTCCCAGGCACTCACCCTGGTCAT | |
| | CCCCAAAACTCCCCAAAAAAGTCCAGACTGTGAGGAGCCGTCC | |
| | TCTGTCTCCTGTGGCCACACCTCCTCCTCCTCCCCCTCCTCCT | |
| | CCTCCTCCCCCTCCTTCTTCCCAAAGGCTGCCACCACCTCCTC | |
| | CTCCTCCCCCTCCTCCACTCCCAGAGAAAAAGCTCATTACCAG | |
| | AAACATTGCAGAAGTCATCAAACAACAGGAGAGTGCCCAACGG | |
| | GCATTACAAAATGGACAAAAAAAGAAAAAAGGGAAAAAGGTCA | |
| | AGAAACAGCCAAACAGTATTCTAAAGGAAATAAAAAATTCTCTG | |
| | AGGTCAGTGCAAGAGAAGAAAATGGAAGACAGTTCCCGACCTT | |
| | CTACCCCACAGAGATCAGCTCATGAGAATCTCATGGAAGCAAT | |
| | TCGGGGAAGCAGCATAAAACAGCTAAAGCGGGTGAGTCTATG | |
| | GGACGCTTGATGTTTTCTTTCCCCTTCTTTTCTATGGTTAAGTTC | |
| | ATGTCATAGGAAGGGGATAAGTAACAGGGTACAGTTTAGAATG | |
| | GGAAACAGACGAATGATTGCATCAGTGTGGAAGTCTCAGGATC | |
| | GTTTTAGTTTCTTTTATTTGCTGTTCATAACAATTGTTTTCTTTTG | |
| | TTTAATTCTTGCTTTCTTTTTTTTTCTTCTCCGCAATTTTTACTAT | |
| | TATACTTAATGCCTTAACATTGTGTATAACAAAAGGAAATATCTC | |
| | TGAGATACATTAAGTAACTTAAAAAAAAACTTTACACAGTCTGC | |
| | CTAGTACATTACTATTTGGAATATATGTGTGCTTATTTGCATATT | |
| | CATAATCTCCCTACTTTATTTTCTTTTATTTTTAATTGATACATAA | |
| | TCATTATACATATTTATGGGTTAAAGTGTAATGTTTTAATATGTG | |
| | TACACATATTGACCAAATCAGGGTAATTTTGCATTTGTAATTTTA | |
| | AAAAATGCTTTCTTCTTTTAATATACTTTTTTGTTTATCTTATTTC | |
| | TAATACTTTCCCTAATCTCTTTCTTTCAGGGCAATAATGATACAA | |
| | TGTATCATGCCTCTTTGCACCATTCTAAAGAATAACAGTGATAA | |
| | TTTCTGGGTTAAGGCAATAGCAATATCTCTGCATATAAATATTTC | |
| | TGCATATAAATTGTAACTGATGTAAGAGGTTTCATATTGCTAATA | |
| | GCAGCTACAATCCAGCTACCATTCTGCTTTTATTTTATGGTTGG | |
| | GATAAGGCTGGATTATTCTGAGTCCAAGCTAGGCCCTTTTGCT | |
| | AATCATGTTCATACCTCTTATCTTCCTCCCACAGGTGGAAGTTC | |
| | CAGAAGCCCTGCGATAAAAACATGATCTTTAGAAGAGGATGCA | |
| | GAACTGTTCAGTGGTATTACATGAAATGCATTGTGAGATGTTTC | |
| | TAAAATACCTTCTTCAATTCAAAATGATCCCTGACTTTAAAAATA | |
| | ATCTCACCCATTAATTCCAAAGAGAATCTTAAGAAACAATCAGC | |
| | ATGTTTCTTCTGTAAATATGAAAATAAATTTCTTTTTTATGTCGT | |
| | GAGATTTGTATTGGCAAGAAGCAGTTAATTTAAAGATGCTCTTC | |
| | CTATCTGTGGATGTGTTGGTAACTCCGAGTTGTAATGAGTTCAT | |
| | GAAATGTGCTGTTATTTTTGTAATCTCAATAAATGTGGATTGAA | |
| | GTTTTTTCCCTTTTTTTAAAGCCAAACTAATATTTTTCTGTGACTT | |
| | GATACATCTGTCAGATTTTTGTAATCTCGATAAATGTGTATTGAA | |
| | GTTTTTTCCCTTTTTTTAAAAAGCCAAACTAATATTTTTCTGTGA | |
| | GTTAATACATCTGTCAGGTGTGTATGTAACATTACTGGACATTA | |
| | AAAAAAAATATTACATTCTCACCCAAAAAGGGTTTGGGTCCTAA | |
| | GCATTTGCCTTTCTTTGTTTCCTCTTGTTCAAGAAAATCTGATTA | |
| | GATCTCTTTCTAAAGGACTGCAAGCAATAATTTTTTTTTATATTT | |
| | TATTTATTTATTTATTTTTTTGAGACAAGTCTTGCTCTGTTACCC | |
| | A | |
| LMOD2 gene | ATGTCTACCTTTGGCTACCGAAGAGGACTCAGTAAATACGAAT | 22 |
| (G) | CCATCGACGAGGATGAACTCCTCGCCTCCCTGTCAGCCGAGG | |
| | AGCTGAAGGAGCTAGAGAGAGAGTTGGAAGACATTGAACCTGA | |
| | CCGCAACCTTCCCGTGGGGCTAAGGCAAAAGAGCCTGACAGA | |
| | GAAAACCCCCACAGGGACATTCAGCAGAGAGGCACTGATGGC | |
| | CTATTGGGAAAAGGAGTCCCAAAAACTCTTGGAGAAGGAGAGG | |
| | CTGGGGGAATGTGGAAAGGTAGGCTCTCGGGACTTTTCCTTG | |
| | GCTAACCCCACCTCCCCATCACCCCATCCCAAACCCAGACACT | |
| | TGTTTTCCCTAACTTCTCAATCCTCATCACTTGAATTTTCCTATA | |
| | ACCCATTTATACTTGCTACTAAATCATTTTTCAGGCTGAAATAAT | |
| | CATGTAAATGCTATGGCCACCCAATGGATCATAATATGTAAAAT | |
| | TCTTAATTGGGGCTCTTTGATTGATTACAAATAGAAAGCTCAAG | |
| | AGGAAAATAATTCACAATCAGTGTATGGGATTCAATATTTTGCC | |
| | AAACATAAGTGTTGTTTGAGCTATATGTTGGTAATGTGGCATAA | |
| | TATTTGTTCCCAAGATACATGCATACGTATAAATGTATACTTTTT | |
| | TTTCAGTTAAGTACATTTATTTTCTCCTATAAAGACAAAAGTGGG | |
| | AACGCTGGTTGCTTTGACTGTGATAGTAGCTATGGTGAAATAGT | |
| | TCTTTTCCAATACCAAGAAGTGTGGGGTGGTGGGAAGTACATG | |
| | ACACTGAGAGTCACAGGGCCTTGTTATGAGTCCCTGCTCTGTT | |
| | CCCACATCTTTAGACCTTGAACAATCACTTTTACAGGGTTCTCT | |
| | TTCCCTCTGGACATTGAGGGAGTTAGAGACAGTGTTTCTTTTGC | |
| | GAATCAAGAACCCCTTTGAGATTTTGATAAGTTATGAACCTTCT | |
| | TAAGAAAATAGCCCTGGGCTTGTGCAAAATACACCAACTTTTGC | |
| | ATAGAATTTCAAAATGATAACACATCCTGAAACCCATCTGAGGA | |
| | CTCAGGGGCAGCACAGCTGTATAGATGATCTCTTCGATTTCTG | |
| | TCTCCTTTACGATGGTGATCCTGAGGTTAGGAAATGTGCTTATA | |
| | TCAGAGCTCAGGCCTATAAAGCTCTTGTGTACTGCCCAGTAAA | |
| | CAGTTATTTTGAATTTGCCAAATGAGTCAATACCAGAAGGAAGT | |
| | CCAAAAGACTATTATTCTCTGGGAAAACAGTAATGACAATAAAG | |
| | TGTCCATATAACAAAAAAAAAATTCTTCCGGAATTATGGAAGAA | |
| | CATAGGTCAGGAGAAACAGTTTAAAACCCTTGCACTCTGTTGG | |
| | AATCATCTAAAATCAAAATGGCCTAAAGCCAATGGCAAGACTTC | |
| | CACAGTACTAATCTAGGGCTGTTAGATGATCTTGATGATAGGGT | |
| | TATCTTGGCCACAATTGCAGCAAGATATGTGTGAGTTTTGTCCA | |
| | CGTCAATCATTGCAGGAAGGGCCACTGGGATCTGGCCAGGGT | |
| | CTGCCAAGGCCACAGTACTAATCAGCTAGAAGGGGTGTGTTAG | |
| | AAGGAAGGACACATACAAGTCAGTACTTGGGCAGGTCAATTCC | |
| | TCCACAACCATTGTTTTAAGAGGGATTTTTTTTTCTTTGATGTGA | |
| | TAATGAGCATCAAAATAACAATGACATTATCATCACAATTATTTA | |
| | AAACTCTCTTTAATAAAATAAAACTGGCTGGGTATGCTGGCTCA | |
| | CGCCTATAATTCCCACACTTTTTTGGGAGGCTGAGGCAGGAGA | |
| | ATCACTTGAGCCTAGGAGTTCAAATCCAGCTCTGGCAATATAGT | |
| | GAGATCTCATCTCTACAAAAAAAAAAAAAAAAAAAAAAAATTAG | |
| | CTGGGTTTAGTGGAACATTAGCAGGTGGTAGTCCCAGCTGCTC | |
| | AGGAGGCTGAGGTGGGAGGTTGGCTTCAGCATGGGAGGTTGA | |
| | GGCTGCAGTGAGCTATGGTTGTGCCACTGCACTCAGCCTGAG | |
| | CCACAAACCAAAACCTTGTCTCATTAAAAAAAAAAAAAAAAAAA | |
| | AAGAATAAAACTGTCTCAGAATATCCAGGCTGTACAACCAATGT | |
| | ATCAATAATCCAACAAAGGCTTTTTAATGTCACAATGACATGAT | |
| | AGTGGTGGGAACCAAAGAGCAAAACCTATCGTCAGCCGGACG | |
| | AACCCACCTGATCCTGCCAAAGGTTTTGCTGATCCCTGCCAAT | |
| | GGCTTATCAGCTCAATTAGAGAGGCTGCTGTGTTGATGGACTT | |
| | CGGTTCCTTTCTAGGCTATTAACTTTTTCTCCCAGTACTTTCCCT | |
| | GCTGGCCAGCTGTCTGTAGTTGCCAGGCATGATGAGCAGGAA | |
| | CTTGCTGATGGAGGGTGCAAATCCAATTCCCACAACAGGGAAA | |
| | ATCTTCGAGCAGCTGTTGGGTGGCTGGGGGAGCAAGGTAGTT | |
| | GCGTAAGTGAAGAGCCATAAAGGAAGGACCCTGAGAAGGAGC | |
| | GGAGGGTCTTGATTGATTATCAGGCGGCTGGCTCTGTAAAGGC | |
| | TTATAAAAAGCAGATGCAACACCTGTTGAATGCCAGCCTTTCTG | |
| | CCCACTTGGGCTTACACAAACTTATGCCGCATTGGGGCTCCCA | |
| | GGGACCCTAGAATCCCAATGGTCTAGCTCCCCTATTGAGCAGT | |
| | GGTTAAATCTCAGCACAGGTGCCAATGAGTTTCTCCCATTATCT | |
| | CACTTCATTTTTAACTTTCTGCACTTGAGAACTGAGACAAGGCA | |
| | AGGTTAAGAAACTCATTAGAGGTTAAGAGCTTGTACATAGAAGT | |
| | GAAAATTAAAGAACATTTCAAAATTGTGGGTGCCCGGTCAGCA | |
| | GCATCAGCATTACCTGGGATCTTCTTGGAACTGCAAATTATAGA | |
| | GTTCCCGTCAATCTGATGAATGATAAACTCTGGGGATGGGGCC | |
| | CAACAATTGGTGCTCTCACAGGTGATTATGACACACAGTAAAAC | |
| | TTAAGAACCGCTCAACTACAGTTACCTCCTGAATAACATGGGG | |
| | GTCAGGTGAACCAACTCCTGCCCCTGCAGCATCCAAAATTCAT | |
| | GTATAACTTTTGACTCCCCCAAACCTTAACTACTAATAGCCTAC | |
| | TGTGGACAGAAGCCTTACCAATAACATAAACGGTCAATTAACAC | |
| | ATACTTTGAATGTGATATGTACATACATAATAGGGAGGTGATCA | |
| | GGAGAAACAACCGGCCACTCTTCTCATTACCTGATTTGTTTTCT | |
| | AAGTTTACTAACATTCCCCTTCTCCTTGTCCAGTCCCTTGCCAT | |
| | CTCATCTCCCACCTCCTTATTCTATACCCTCCTTCCCACCTCCA | |
| | TTCTCCAAAGAATCAGGGTTAATGCCAAAGCAGTGGCATATAG | |
| | GAAATCTGTAATAAAACAGGCCCCCTAGGGACAGAGTATTTTAT | |
| | AAAGTAAGCGAAGTCTGTTTTTTGCATCCTCTCACCTTGCCTCT | |
| | GTAGACACCTTTTCCCCTTCTGTTCTTTCTCCAGATGTCCCCCA | |
| | CCTAGGGTGACTAATCATCTCAGATTGTCCGGAACTATCTAGAT | |
| | TTCATGACTAAAATCCTGGGAAACCCTTCACACTCAGGCAAACT | |
| | GGGAGGGTTTATCACCTTTCTCCTGTGTGTAGTCCTGACTCTC | |
| | AGCTGCTGTTAACCAATGTCAACTTAGAGCCATTCAGTAAGCAA | |
| | ATAATTATTGAGCTCCTACTACATGCTAAGCACTGTGCTAGGTG | |
| | AGCAAAGCAGACATGGTCCACTGCTCTCAACAGCTAACAGTCT | |
| | GCTGGGAAAAACAAGTGACCAAAATAAAGTGCGACAGGTACTT | |
| | TATTGGGGGTGGGTACAATGGTATGGGAATGCGTCTGAGGGA | |
| | TATTTGGAGAGGATGTCAAAGAAGGCTTTCTGGGAGATCTATC | |
| | ATTTAGATGCTATATGACAATTCACAGTTTCAGATATTATCTGTC | |
| | CAGGTACATTATAAGGGGAAAACAGATCTTGCAGATAGAGCCA | |
| | GAGGGTCTTTAAAAAACCAAAGGGAGTAAAATGTAATGGTGTC | |
| | ATACAAGGCCGTATACCTTGGATTTTGAGACAGGCCCCTTTCC | |
| | TCCAAATACTGAAACAGCTAAAAATACATACAAACACATGAACA | |
| | CACACAGCTTTACAGGGACTAGAATTATAAGGAATTTGGAGTG | |
| | GAATGGGGTGAGGAGAAGTCAGCCCACAATTCTTTCCTTCCTC | |
| | GGGTTAATCCATTTGTTTCACTGCTGGACAGGGAAGGCTTCCT | |
| | CTCTTCCCTCACGTATGCCTGGAGACTTGGCTAGAGCCACACT | |
| | CTTTTTCTTATGTTGTTGCATTGTGACTTGCAGCCAGCTGCCAC | |
| | CAAACATTTCTCTCTCTTTCTCTCTCTCTCTCTCTCTCCCTCTCT | |
| | CTCTCTCTCTCACACACACACACACACACAGAGACACACAGAG | |
| | TATCTCTCTCTCTCCCTCCCTGCTCCTGAAGAAGTGAATAAATT | |
| | AAGACTGGAAAAGTCATCCCAGGAGTGGCTGACCCCTTCCCCA | |
| | GGCCATCACTCGGTGCCTGCCACTCCAAACAGTGGATAACTGG | |
| | AGGCTATTTTGTGATAGCAACTGGGCAAACGAACCTGGAGACA | |
| | AGGGCAGTGTTTTCCAAAGCTAGATCTACATGCTTCAGGCAGC | |
| | GGGCGCCCTCGAGGTCAAAAAAAAAAAAGTTGCCAATATAGAG | |
| | AGAAGAATCAGGGCCTGGAGGACAGGGCAGGGTCCCAGGGC | |
| | TGTAGAGAAGATAAAGAGAAAGGACTAAAAAGACAGCAGGAGA | |
| | AAAAGATTAGACTCTGTCAGCAATTTCTAGACCTTTTTTTTTTTT | |
| | AATCAGAAAAGATAGCAAGAAAAAGAGAAACAAGGGGTCACTT | |
| | GAGCGTCTTTGTACAGCAAATCTTGTAAACAGGAAAGACAAAG | |
| | ATAGAATGAAATTGTGTTTGCCTGAAAGCCTTTCATAATTGATA | |
| | CATGCTAAATAATTTAAGACCTTCAGGCTGGGCAATGGCTTACT | |
| | TCAAGATTTTCTGCTGTTCTCTGCTGTTCCATCCACTTTACTGAT | |
| | TAGAATCTAGTGGATGCCAAGGGTGATTATCTTTCCCACCTGG | |
| | TCTTCTACAGTCTGTCTCATTTAACCCTGGACTAGTTGGAGGTT | |
| | TCAAGTTGATTTTCCCAAAAGGGTGACCTGAATGTTTTCTCTGC | |
| | ATCGAACTGGATTTTGACCTCCTGCTGTCCTGAGCAGCAGCCC | |
| | TGCCTTTAGGCTTCCCTTGCCCAGATAAGGGACAGCTACAGAG | |
| | GCAGCTCTCAGGTGATTCAGAGAGTCTTCTGTGGTAGAGCCCT | |
| | GTGCCGGGCTCTACCGTAGAAGCAGATAAATAAATGCTGGAGG | |
| | AGCAAATGACAGCTCTGCTTCTGTCTACACTGAGGCAAGGCCT | |
| | GGCAGAGAGTGGTTGGCTTCTGTCACATCTGCTATGGATGGAA | |
| | AATTTGGGAATTTTTGAATTAAAAAGGGAAACTCATTAACATATT | |
| | GTATATTTTTGTTGTCATTTGATTGCTTGAGATGGAGAAATAAG | |
| | AAAGAACAGAGTGATTCCAGAAGGGAGGGTTGAATTTGCTACA | |
| | TTGTAAGTAAATGGAGAGTAAACCAGTAAGAGTCGGTCCTCAG | |
| | GAGGTGAACAAATTTATCAAGGGTTTTCCAAAGCTGAAGTTCTG | |
| | ATTTATTTGTTCAAATTAGCAAAATGATTATATATTTTTATTCAAT | |
| | AAGTTGAATTCTGGATATTCTGCAGAAAACAAGAGAAATGTTTG | |
| | TAGAAAAAGCTATTGCTATTTATTGCAATAAAATGTCAGCCTTG | |
| | GTCATGAGAACTAGACAGAATGGAAGCAGAGGATTATTAACTTT | |
| | TTTTATTATAAACTTATGTCACTTGCCATGTGCTACTTTTGCAAG | |
| | TTAAAAAATGTACTTAAAAATGGTATCATTTTTAAGAAGCTTAAT | |
| | GATGATATCATACTCTTTAGGTTGCAGAAGACAAAGAGGAAAGT | |
| | GAAGAAGAGCTTATCTTTACTGAAAGTAACAGTGAGGTTTCTGA | |
| | GGAAGTGTATACAGAGGAGGAGGAGGAGGAGTCCCAGGAGGA | |
| | AGAGGAGGAAGAAGACAGTGACGAAGAGGAAAGAACAATTGA | |
| | AACTGCAAAAGGGATTAATGGAACTGTAAATTATGATAGTGTCA | |
| | ATTCTGACAACTCTAAGCCAAAGATATTTAAAAGTCAAATAGAG | |
| | AACATAAATTTGACCAATGGCAGCAATGGGAGGAACACAGAGT | |
| | CCCCAGCTGCCATTCACCCTTGTGGAAATCCTACAGTGATTGA | |
| | GGACGCTTTGGACAAGATTAAAAGCAATGACCCTGACACCACA | |
| | GAAGTCAATTTGAACAACATTGAGAACATCACAACACAGACCCT | |
| | TACCCGCTTTGCTGAAGCCCTCAAGGACAACACTGTGGTGAAG | |
| | ACGTTCAGTCTGGCCAACACGCATGCCGACGACAGTGCAGCC | |
| | ATGGCCATTGCAGAGATGCTCAAAGTCAATGAGCACATCACCA | |
| | ACGTAAACGTCGAGTCCAACTTCATAACGGGAAAGGGGATCCT | |
| | GGCCATCATGAGAGCTCTCCAGCACAACACGGTGCTCACGGA | |
| | GCTGCGTTTCCATAACCAGAGGCACATCATGGGCAGCCAGGT | |
| | GGAAATGGAGATTGTCAAGCTGCTGAAGGAGAACACGACGCT | |
| | GCTGAGGCTGGGATACCATTTTGAACTCCCAGGACCAAGAATG | |
| | AGCATGACGAGCATTTTGACAAGAAATATGGATAAACAGAGGC | |
| | AAAAACGTTTGCAGGAGCAAAAACAGCAGGAGGGATACGATG | |
| | GAGGACCCAATCTTAGGACCAAAGTCTGGCAAAGAGGAACACC | |
| | TAGCTCTTCACCTTATGTATCTCCCAGGCACTCACCCTGGTCAT | |
| | CCCCAAAACTCCCCAAAAAAGTCCAGACTGTGAGGAGCCGTCC | |
| | TCTGTCTCCTGTGGCCACACCTCCTCCTCCTCCCCCTCCTCCT | |
| | CCTCCTCCCCCTCCTTCTTCCCAAAGGCTGCCACCACCTCCTC | |
| | CTCCTCCCCCTCCTCCACTCCCAGAGAAAAAGCTCATTACCAG | |
| | AAACATTGCAGAAGTCATCAAACAACAGGAGAGTGCCCAACGG | |
| | GCATTACAAAATGGACAAAAAAAGAAAAAAGGGAAAAAGGTCA | |
| | AGAAACAGCCAAACAGTATTCTAAAGGAAATAAAAAATTCTCTG | |
| | AGGTCAGTGCAAGAGAAGAAAATGGAAGACAGTTCCCGACCTT | |
| | CTACCCCACAGAGATCAGCTCATGAGAATCTCATGGAAGCAAT | |
| | TCGGGGAAGCAGCATAAAACAGCTAAAGCGGGTAAGTAACCA | |
| | GAGAACAGACATAGGGGCACAGATAAAGTAAATGAGTTGTCCT | |
| | CCATTGCATGGTGGTACCAAAGTCACCTCTCACAATACTTATCA | |
| | ATACTTTCAATATTTTAGTATGCGAGAGCAAACACACCAAGTTT | |
| | GAAACATTAGGAGCAGGCACACAAGTGAGCACATTTCTATTTG | |
| | AGAGGAACGCCTGGGCCGCTTTCCCAGCCTCTCAATCATATAA | |
| | GGGCAAGGACCATTTTCATACACGTCCCAATTCCCTTAAGAAG | |
| | AAAACCAGGGAATATAATCTAAATTCCAAAAGGATTCACCACTT | |
| | GTCAAAAATTTTACCACAGAGTTGTGACTGATTCGATACCTAAT | |
| | TTATAACATAAAATTTATAATGTGGTAAAAAAATTACCATGCAGC | |
| | AATAATCAACCTGAGTTCTTCTCAGAGGATACTGCTAGAGACAT | |
| | ATCCTACAGATATTTTTCACTTATCATGTGTGTTGTTTCATTGAG | |
| | AGAAAATCCGCTATTTTTGTAGGTGGAAGTTCCAGAAGCCCTG | |
| | CGATAAAAACATGATCTTTAGAAGAGGATGCAGAACTGTTCAGT | |
| | GGTATTACATGAAATGCATTGTGAGATGTTTCTAAAATACCTTC | |
| | TTCAATTCAAAATGATCCCTGACTTTAAAAATAATCTCACCCATT | |
| | AATTCCAAAGAGAATCTTAAGAAACAATCAGCATGTTTCTTCTG | |
| | TAAATATGAAAATAAATTTCTTTTTTATGTCGTGAGATTTGTATT | |
| | GGCAAGAAGCAGTTAATTTAAAGATGCTCTTCCTATCTGTGGAT | |
| | GTGTTGGTAACTCCGAGTTGTAATGAGTTCATGAAATGTGCTGT | |
| | TATTTTTGTAATCTCAATAAATGTGGATTGAAGTTTTTTCCCTTT | |
| | TTTTAAAGCCAAACTAATATTTTTCTGTGACTTGATACATCTGTC | |
| | AGATTTTTGTAATCTCGATAAATGTGTATTGAAGTTTTTTCCCTT | |
| | TTTTTAAAAAGCCAAACTAATATTTTTCTGTGAGTTAATACATCT | |
| | GTCAGGTGTGTATGTAACATTACTGGACATTAAAAAAAAATATT | |
| | ACATTCTCACCCAAAAAGGGTTTGGGTCCTAAGCATTTGCCTTT | |
| | CTTTGTTTCCTCTTGTTCAAGAAAATCTGATTAGATCTCTTTCTA | |
| | AAGGACTGCAAGCAATAATTTTTTTTTATATTTTATTTATTTATTT | |
| | ATTTTTTTGAGACAAGTCTTGCTCTGTTACCCA | |

Steric-blocking oligonucleotides: 20-22 mer oligonucleotides uniformly modified with a phosphorothioate backbone and 2′-O-methoxy-ethyl (2′-MOE) bases were synthesized by Integrated DNA Technologies. The sequences, which are named according to their location upstream of the junction between exons 2 and 3 of LMOD2, are in Table 3.

TABLE 3

| Steric-blocking | | |
|---|---|---|
| Oligonucleotide | Sequence | SEQ ID NO: |
| L2[−23] | GCTTTAGCTGTTTTATGCTGC | 23 |
| L2[−24] | GCTTTAGCTGTTTTATGCTGCT | 24 |
| L2[−25] | CTTTAGCTGTTTTATGCTGCTT | 25 |
| L2[−26]: | TTTAGCTGTTTTATGCTGCTTC | 26 |
| L2[−27] | AGCTGTTTTATGCTGCTTCC | 27 |
| L2[−28] | GCTGTTTTATGCTGCTTCCC | 28 |
| L2[−29] | CTGTTTTATGCTGCTTCCCC | 29 |
| L2[−30]: | TGTTTTATGCTGCTTCCCCG | 30 |
| L2[−31] | GTTTTATGCTGCTTCCCCGA | 31 |
| L2[−32] | TTTTATGCTGCTTCCCCGAA | 32 |
| L2[−33] | TTTATGCTGCTTCCCCGAAT | 33 |
| L2[−34]: | TTATGCTGCTTCCCCGAATT | 34 |
| L2[−35] | TATGCTGCTTCCCCGAATTG | 35 |
| L2[−36] | ATGCTGCTTCCCCGAATTGC | 36 |
| L2[−37] | TGCTGCTTCCCCGAATTGCT | 37 |
| L2[−38]: | GCTGCTTCCCCGAATTGCTT | 38 |
| L2[−39] | CTGCTTCCCCGAATTGCTTC | 39 |
| L2[−40] | TGCTTCCCCGAATTGCTTCC | 40 |
| L2[−41] | GCTTCCCCGAATTGCTTCCA | 41 |
| Scrambled (Scr): | TCTAACTACGCCTTGGTTTC | 42 |

Western blot analysis: Cells were washed twice with PBS and collected with a cell lifter in ice-cold lysis buffer (150 mM NaCl, 1.5 mM MgCl2, 1 mM EGTA, 10 mM sodium pyrophosphate, 10 mM sodium fluoride, 0.1 mM sodium deoxycholate, 1% Triton X-100, 1% SDS, 10% (vol/vol) glycerol, 25 mM HEPES, pH 7.4, plus 1× Halt Protease Inhibitor Cocktail [ThermoFisher Scientific) 78439]) and sonicated 4×10 sec on power 2. Samples were spun down for 15 min at 16,000×g at 4° C., flash frozen, and stored at −80° C. until processing. Total lysate protein concentration was normalized by BCA assay (ThermoFisher Scientific, 23225), and samples were incubated in 1× Laemmli sample buffer at 100° C. for 10 min. 20-30 g of total protein was resolved on a 10% SDS gel, transferred to PDVF, and the membrane stained with Ponceau S. The membrane was then scanned and allowed to dry. For probing, the membrane was blocked with 5% (wt/vol) nonfat dried milk/TBS for 1 h at room temperature followed by incubation with primary antibodies in 1% BSA (wt/vol)/TBST overnight at 4° C. Primary antibodies included: mouse monoclonal anti-GFP (0.2 g/ml; B-2, Santa Cruz Biotechnology, sc-9996), rabbit polyclonal anti-Lmod2 (0.1 g/ml; E13, Santa Cruz Biotechnology, sc135491); rabbit polyclonal anti-EIF4A3 (0.2 g/ml; Proteintech®, 17504-1-AP); mouse monoclonal anti-MAGOH (0.4 g/ml; F-6, Santa Cruz Biotechnology, sc-271365); mouse monoclonal anti-vinculin (1:6000; hVIN-1, Sigma, V9131) and mouse monoclonal anti-GAPDH (1 g/ml; Proteintech®, 60004-1). The membranes were then washed 5×5 min in TBST and incubated with Alexa Fluor 680 or Alexa Fluor 790 AffiniPure donkey anti-rabbit or anti-mouse IgG (1:40,000; Jackson ImmunoResearch) diluted in 5% milk/TBST for 1 h at room temperature. Following 5×5 min washes in TBST, blots were imaged and analyzed using a LI-COR Odyssey CLx imaging system (LI-COR).

Reverse transcription-quantitative polymerase chain reaction: RNA was extracted from HEK293 cells using a Quick-RNA Miniprep Kit, including an on-column DNase digestion, according to the manufacturer's instructions (Zymo Research). cDNA was synthesized from 500 ng of total RNA using RevertAid™ RT or TaqMan™ Reverse Transcription Kits (ThermoFisher Scientific, K1691 or N8080234) with oligo deoxythymidine (dT) or random hexamer primers. Five microliters of template cDNA (diluted 1:25) were used in a PCR with Maxima SYBR Green qPCR master mix (ThermoFisher Scientific, K0252) on a LightCycler® 480 (Roche). To determine relative gene expression, the Ct method was used. Each cycle threshold value (Ct) was normalized to that of a reference gene (ΔCt) and then compared with the ΔCt of a control group (typically wild-type LMOD2 CDS or MG) (ΔΔCt). The mean 2\hat{ }-ΔΔCt for each group was then calculated (relative expression). The neomycin resistance (Neo) gene, which is expressed from the same plasmid as the gene constructs (pcDNA3.1 [−]) was used as the reference gene for transient transfections. The hygromycin resistance (Hyg) gene, which was inserted into the genome along with the LMOD2 constructs, was used as the reference gene for the stable expression cell lines. The ribosomal protein L32 (RPL32) was used as the reference gene for the RNA-IP experiments since it has multiple exons and introns and is therefore predicted to associate with the exon junction complex. Primer efficiencies were determined from standard curves generated using serial dilutions of cDNA. To assess mature LMOD2 mRNA levels, primers spanning introns were used in conjunction with cDNA amplified with an oligo dT primer. To determine LMOD2 pre-mRNA levels, primers within intron 2 were used in conjunction with cDNA amplified with a random hexamer primer.

Luciferase Assays: A pmirGLO Dual-Luciferase miRNA Target Expression Vector (Promega, E1330) was modified to remove the stop codon from firefly luciferase. LMOD2 CDS and MG constructs (with and without disease-causing mutations) were inserted in-frame downstream of firefly luciferase to generate fusion proteins. AD-293 cells were plated in 24-well culture dishes and transfected the next day (at 50-80% confluent) with luciferase-LMOD2 fusion constructs and/or SBOs using Lipofectamine 3000 Transfection Reagent with a lipid to DNA ratio of 2:1 according to the manufacturer's instructions (Invitrogen, L3000015). Two days later the cells were dissociated with a small volume of 0.25% trypsin (Gibco, 25200-056) and quenched with media in a final volume of 100 L. 75 L of cells were then added to white opaque 96-well plates (Corning, 353296) and the Dual-Glo Luciferase Assay performed according to the manufacturer's instructions (Promega, E2920). Firefly and Renilla luminescence were measured on a GloMax®-Multi Detection System (Promega).

RNA Immunoprecipitation: RNA Immunoprecipitation was performed as previously described with some modifications. Briefly, an isogenic stable HEK293 cell line expressing the LMOD2 minigene containing the patient's mutation (W398*) was transfected 2 days after plating with 100 nM of SBOs. The cells were washed 2× with ice-cold PBS on ice, collected in Polysome Lysis Buffer (PLB-100 mM KCl, 5 mM MgCl2 10 mM HEPES-NaOH pH 7, 0.5% Nonident P-40, 1 mM dithiothreitol, RNase Inhibitor-Murine [1 U/μl; New England Biolabs, M0314L], 1× Halt Protease Inhibitor Cocktail [ThermoFisher Scientific, 78439]) with a cell lifter, pipetted to break up clumps, incubated on ice for 5 min and stored at −80° C. After thawing, the lysate was centrifuged at 16,000×g for 15 min at 4° C. The supernatant was precleared with 20 μl of resuspended Protein A/G PLUS-Agarose (Santa Cruz Biotechnology, sc-2003) plus 2 μg of Normal Rabbit IgG (Cell Signalling, 2729) for 1 h at 4° C. After centrifugation, the protein concentration of the supernatant was determined by BCA assay (ThermoFisher, 23225). To immobilize antibodies, 15 μg of rabbit polyclonal anti-eIF4AIII/EIF4A3 (ThermoFisher, A302-980A) or Normal Rabbit IgG was added to 20 μl of resuspended Protein A/G PLUS-Agarose in 100 μl of NT-2 buffer (50 mM Tris-HCl pH 7.4, 150 mM NaCl, 1 mM MgCl2, 0.05% NP-40) and incubated with rotation for 1 h at room temperature. Following 6 washes with NT-2 buffer, the antibody-bound beads were added to 2 mg of protein lysate in a total volume of 1 mL of PLB supplemented with 25 mM EDTA and incubated with rotation overnight at 4° C. After 6× washes with NT-2 buffer, a sample of the immunoprecipitate was processed for western blot analysis. The remaining immunoprecipitate was resuspended in 150 μL of Proteinase K buffer (1×NT-2 buffer+1% sodium dodecyl sulfate) with 1.2 mg/ml of Proteinase K and incubated for 30 minutes at 55° C. RNA was then extracted using phenol-chloroform and quantified with a Qubit RNA HS Assay Kit (ThermoFisher, Q32852) and fluorometer. cDNA was synthesized using random hexamer primers from 100 ng of RNA as outlined above.

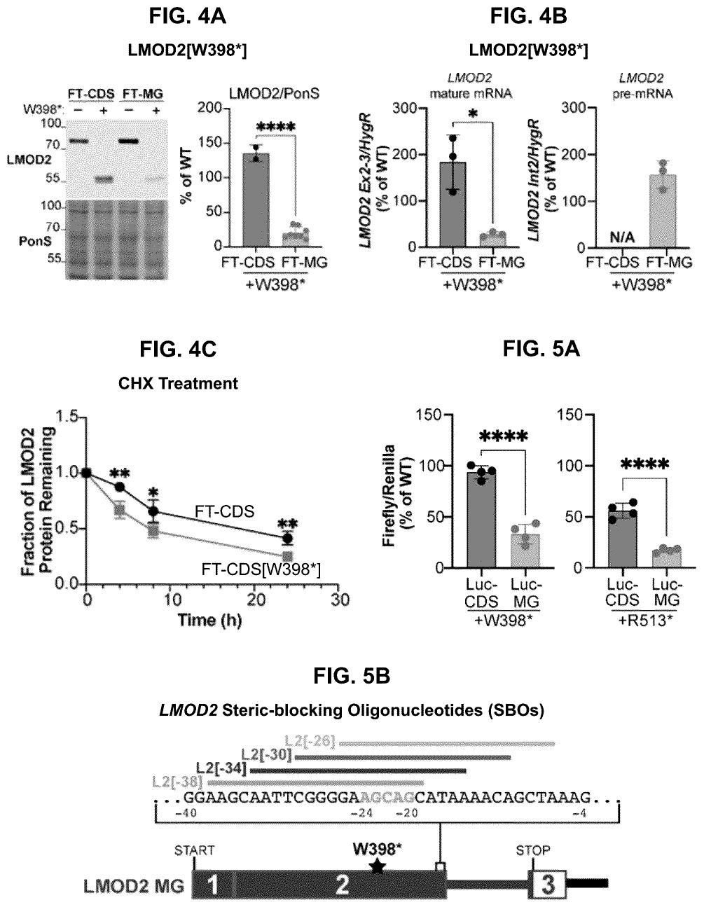

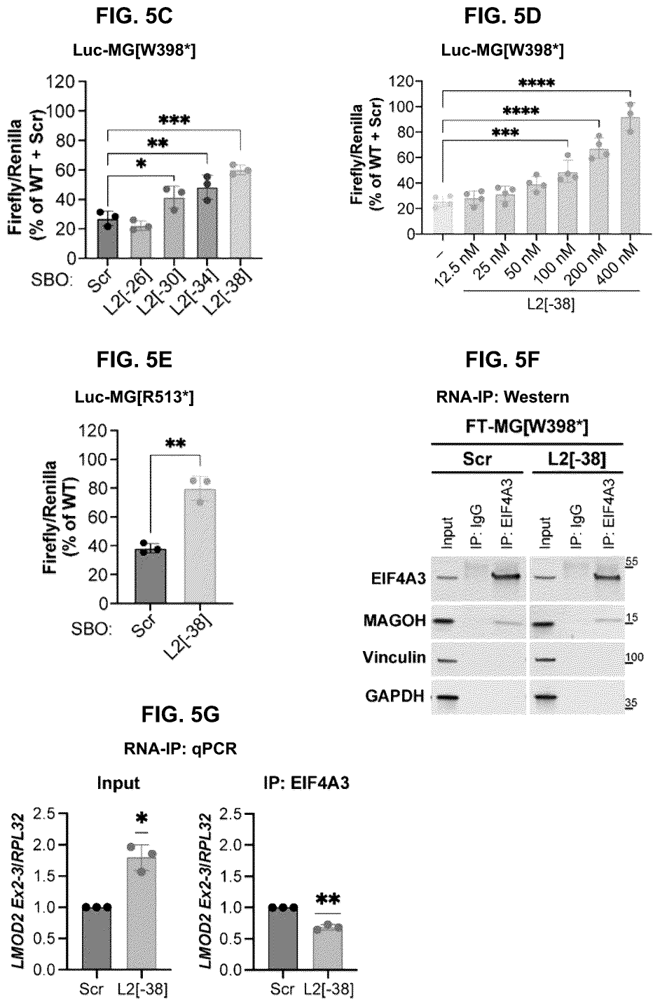

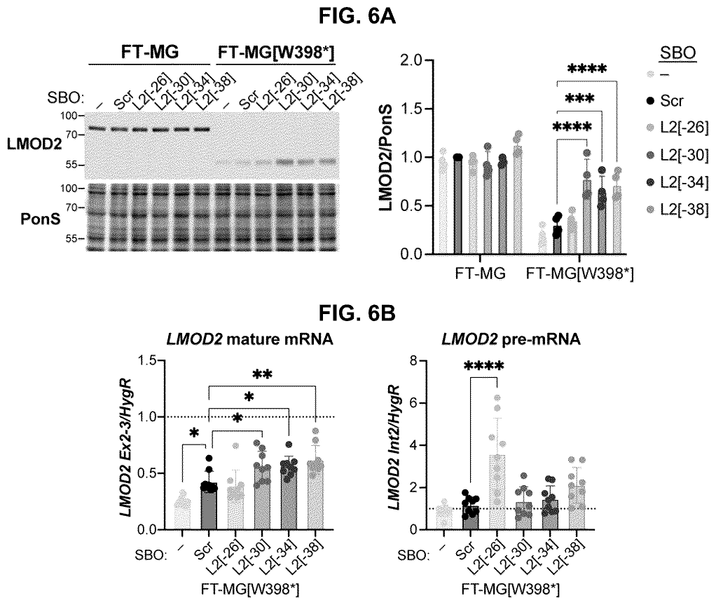

Statistical Analyses: All statistical analyses were performed using GraphPad Prism version 10.0 for macOS (GraphPad Software Inc.). Two groups were compared using Student's t-tests (FIG. 1A-1D, FIG. 3A-3B, FIG. 4A-4C, and FIG. 5A-5G). Multiple groups were compared using one-way ANOVAs with Tukey's (FIG. 1A-1D) or Dunnett's (FIG. 5A-5G and FIG. 6A-6B) multiple comparison tests. p<0.05 was considered significant. *P<0.05, **P<0.01, ***P<0.001, ****P<0.0001.

The human LMOD2 gene consists of 3 exons and 2 introns. In order to study the effects of disease-causing mutations on LMOD2 mRNA and protein levels human LMOD2 gene constructs consisting of the coding sequence plus both introns (gene [G]), the coding sequence plus intron 2 only (minigene [MG]), and the coding sequence alone (CDS) were generated (FIG. 1A). Protein expression of constructs with and without patient mutations (FIG. 2) was analyzed in a human cell line (AD-293) that does not express detectable levels of LMOD2 protein via western blot analysis. Introduction of a disease-causing mutation (W398*) into the LMOD2 CDS construct results in a truncated protein whose expression level is comparable to that of wild-type CDS (FIG. 1B). Note, that the truncated protein sometimes resolves as two closely sized bands. However, the mutation results in a significant decrease in LMOD2 protein levels when introduced into constructs containing introns (MG and G) (˜20-30% of wild-type [WT] levels, FIG. 1B). Protein levels are reduced to the same extent in the construct with both introns and the construct without intron 1, indicating only the presence of intron 2 is necessary for loss of mutant protein. To determine if there are specific regulatory sequences within intron 2 responsible for regulating the levels of LMOD2, intron 2 was replaced with the second intron of the human-globin gene. The reduction of mutant protein levels was similar regardless of the sequence of intron 2 (FIG. 1B).

Next, two other LMOD2 mutations were tested that lead to DCM in humans. LMOD2 R513* resulted in a significant decrease in mutant protein levels only when present in the MG construct (˜25% of WT levels, FIG. 1C), while LMOD2 V415fs*108 mutant levels were similar to WT in the MG construct (˜90% of WT levels, FIG. 1D). However, the latter mutation resulted in an increase in protein levels when inserted into the CDS construct (˜130% of WT levels, FIG. 1D), resulting in a significant difference in the relative mutant protein levels in the CDS vs MG constructs. These results suggest that the V415fs*108 mutation leads to increased stability of mutant protein and both mutants undergo nonsense-mediated mRNA decay (NMD), although the R513* mutant to a much larger degree.

To determine whether the decrease in mutant LMOD2 protein levels corresponds to a reduction in LMOD2 transcript levels, reverse transcriptase quantitative polymerase chain reaction (RT-qPCR) was employed. The addition of W398* and R513* mutations results in a decrease of LMOD2 MG mature mRNA levels but has no effect on LMOD2 CDS mature mRNA levels when compared to their respective WT constructs (FIG. 3A). Mutant LMOD2 MG pre-mRNA levels are not significantly different than WT pre-mRNA levels (FIG. 3A). This indicates that loss of LMOD2 is occurring post-transcriptionally. Activation of NMD requires an initial round of translation, therefore blocking translation should result in the accumulation of transcripts containing PTCs. To test for this, AD-293 cells expressing LMOD2 MGs were treated with and without the W398* or R513* mutations with 100 g/mL of the translation inhibitor cycloheximide (CHX) for 4-5 h. RT-qPCR analysis revealed an increase in mature mRNA levels following treatment with CHX vs vehicle (VEH) alone, while pre-mRNA levels remained unchanged (FIG. 3B). These data are consistent with NMD as the mechanism underlying the decrease in mutant transcript levels.

In some instances, the efficiency of NMD is decreased for transcripts expressed via transient transfection compared to the same construct stably expressed. Therefore, to determine if the initial results are an underestimate of the degree to which mutant LMOD2 mRNA is degraded, cell lines stably expressing LMOD2 CDS and MG constructs were generated with and without the W398* mutation (Flp-In™ TREx-HEK293 [FT-293] cells). Western blot analysis indicated a nearly equivalent reduction in LMOD2 [W398*] protein as found following transient transfection (FIG. 4A). However, the stably expressing cells displayed a larger decrease in mutant LMOD2 MG mature mRNA levels compared to that observed with transient transfection (FIG. 4B), while the pre-mRNA levels were not changed (FIG. 4B). To determine if the decrease in the levels of mutant LMOD2 protein is solely due to loss of transcript by NMD or if the stability of LMOD2 protein is affected by the W398* mutation, a cycloheximide (CHX) chase experiment was conducted. FT-293 cells stably expressing the LMOD2 CDS, with or without the W398* mutations were treated with CHX, and the levels of LMOD2 protein were monitored over time via western blot analysis. The CDS constructs were used to avoid the loss of mutant protein due to NMD. There was a reduction (˜25-40%) in LMOD2 [W398*] protein at all time points measured compared to wild-type LMOD2 (FIG. 4C), suggesting that, in addition to NMD, a decrease in stability contributes to the loss of mutant protein.

Next, NMD was blocked in an attempt to recover mutant LMOD2 protein. In order to more efficiently screen for protein recovery, a Dual-Glo Luciferase Assay System was utilized. The LMOD2 CDS and MG constructs (with and without the W398* mutation) were fused to the C-terminus of firefly luciferase (Luc-CDS and Luc-MG). Renilla luciferase is also expressed from the same plasmid under the control of a separate promoter in order to control for transfection efficiency and cell death. AD-293 cells were transfected with Luc-CDS and Luc-MG constructs and the ratio of firefly: Renilla luminescence was determined. The W398* mutation results in a decrease in luciferase activity when inserted into the Luc-MG construct (˜70%), but very little change when present in the Luc-CDS construct (FIG. 5A). The 513* mutation also results in a decrease in luciferase activity when inserted into both the Luc-MG and Luc-CDS constructs, but more so in the MG construct (˜40% vs. 80%, respectively) (FIG. 5A). Thus, the luciferase assay provides a system to easily measure LMOD2 protein loss due to NMD. In order to inhibit NMD specifically for LMOD2, the deposition of the exon junction complex (EJC) was blocked only on LMOD2 mRNA. Canonical EJC binding sites are located 20-24 base pairs upstream of the exon-exon junction, and binding is seemingly independent of sequence identity at that location. Four overlapping 20-22 mer steric-blocking oligonucleotides (SBOs; each offset by 4 nucleotides) uniformly modified with phosphorothioate backbones and 2′-O-(2-methoxyethyl) (2′-O-MOE) sugars that span the putative EJC binding site between exons 2 and 3 of LMOD2 were designed; as a control, a scrambled sequence was also generated (FIG. 5B). The modifications provide the SBOs with increased nuclease resistance and improved binding affinities and prevent knockdown of the targeted transcript via RNaseH-mediated cleavage of the SBO-RNA duplex]. 100 nM of each SBO was cotransfected with Luc-MG [W398*] into AD-293 cells. Treatment with three of the SBOs (L2 [−30], L2 [−34], and L2 [−38]) resulted in a significant increase in normalized firefly luciferase activity (FIG. 5C). The most effective SBO (L2 [−38]) displays dose-dependent activity with a nearly full recovery of mutant protein levels to that of WT levels at the highest concentration tested (FIG. 5D). Western blot and RT-qPCR analysis confirmed that the SBOs result in an increase in mutant protein and mature mRNA levels, respectively (FIG. 6). Transfection of L2 [−38] SBO also increases expression of LMOD2 [R513*] mutant protein (FIG. 5E). Thus, SBOs that are effective at recovering mutant LMOD2 protein were identified.

To assess whether the L2 [−38] SBO does indeed block the deposition of the EJC on LMOD2 mRNA RNA-IP (RIP) was used. A core component of the EJC (EIF4A3) was immunoprecipitated from FT-293 cells stably expressing LMOD2 MG [W398*] (FIG. 5F). Probing for another component of the EJC, MAGOH, indicated that the greater complex was pulled down. RNA associated with the complex (or in the input lysate) was isolated, cDNA was generated, and RT-qPCR was used to quantify transcript levels. Analysis of RIP input lysate revealed an increase in LMOD2 MG [W398*] transcript levels following treatment with SBO L2 [−38] compared to treatment with a scrambled SBO, consistent with inhibition of NMD (FIG. 5G). The amount of LMOD2 transcript associated with the EJC is significantly decreased following treatment with SBO L2 [−38] compared to a scrambled SBO (FIG. 5G), indicating that the SBO interferes with the deposition of the EJC on LMOD2 mRNA.

To date, four distinct LMOD2 mutations have been discovered in individuals from five separate families that all present with early-onset dilated cardiomyopathy. The mutations are biallelic in all individuals, with four homozygous for the same mutation and one consisting of compound heterozygous mutations. By analyzing explanted hearts, two of the mutations (p.W398* and c. c.273+1G>A*) result in a lack of full-length or mutant protein expression. The p.W398* mutation, and an additional unstudied mutation (p.R513*), result in very little mutant LMOD2 protein expression when expressed from gene constructs in a human cell line, while another unstudied mutation (p.V415fs*108) does not significantly decrease mutant protein levels. Interestingly, a patient homozygous for the latter mutation displayed the latest onset of disease to date, presenting at 9 months of age, revealing a potential correlation between mutant LMOD2 protein levels and severity of DCM. While the L415Vfs*108 mutation did not result in a significant decrease in mutant protein when expressed from the minigene construct (˜90% of wild type levels), when expressed from the CDS construct the mutation results in an increase in protein levels (˜130% of wild type) suggesting that the mutant protein, which contains 108 random residues C-terminal to valine 415, is more stable than wild type LMOD2.

Multiple lines of evidence point to nonsense-mediated mRNA decay (NMD) as the underlying cause of loss of LMOD2 protein due to the W398* and R513* mutations. First, the mutations only result in a large decrease in protein levels when expressed from constructs containing an intron. This indicates that significant loss of mutant protein is not due to a defect at the level of the protein. Second, mature mutant mRNA levels are decreased, while pre-mRNA levels are unaffected, indicating that the mutation acts post-transcriptionally. Third, mature mutant mRNA levels are increased upon short-term inhibition of protein synthesis with cycloheximide. This is consistent with the requirement of translation to initiate NMD. Fourth, inhibition of a known mechanism through which NMD functions (the presence of an exon junction complex downstream of a premature termination codon) partially recovers mutant protein levels.

Various strategies have been utilized to inhibit NMD including RNA interference to knock down the expression of NMD factors, the use of small molecule inhibitors to directly disrupt the function of NMD factors or indirectly affect cellular function, and steric-blocking oligonucleotides to block the binding of the EJC. Since NMD is known to regulate many endogenous transcripts, the therapeutic potential of general inhibition of NMD may be limited. Thus, steric-blocking oligonucleotides (SBOs) were chosen to take advantage of the specificity of Watson-Crick base pairing to only target the LMOD2 transcript by blocking a single EJC binding site downstream of the PTC. Three of the four SBOs (L2 [−30], L2 [−34], and L2 [−38] effectively increased mutant LMOD2 protein expression. One SBO did not recover mutant LMOD2 protein or mature mRNA levels and resulted in an apparent increase in LMOD2 pre-mRNA levels. The reason for the increase in pre-mRNA levels is not clear but could result from altered pre-mRNA processing and/or degradation.

All of the LMOD2 disease-causing mutations (should they be expressed) result in proteins missing varying degrees of their C-termini, which includes one of the three known actin-binding sites of LMOD2. Thus, while recovery of mutant protein expression, due to successful inhibition of NMD, may improve cardiac function and could potentially provide the patient with time in which to receive other therapies or a cardiac transplant, it is not expected to provide a cure since it is likely that fully-functional protein is not made. Supporting this conclusion, mice expressing the homologous mutation (p.W405*) that was first discovered to cause disease in humans (p.W398*) live significantly longer than Lmod2 knockout mice, but display dilated cardiomyopathy and die prematurely as adults. Alternatively, the combination of SBO treatment with drugs that promote translational readthrough of premature termination codons could prove effective. Greater than 50 compounds have been discovered that promote stop-readthrough and are in various stages of therapeutic development (such as the aminoglycoside gentamicin and the oxadiazole Ataluren). However, the degradation of mutant transcripts containing premature termination codons by NMD could limit the effectiveness of stop-readthrough drugs. Thus, treatment with an SBO that inhibits NMD in combination with a drug that promotes translation of the full-length protein (such as the aminoglycoside gentamicin or the oxadiazole compound Ataluren) could result in a rescue of cardiac function in patients with certain mutations in LMOD2.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method of treating a subject suffering from cardiomyopathy using a steric-blocking oligonucleotide (SBO), the method comprising:
a) determining whether the subject carries a nonsense mutation in the LMOD2 gene by:
i) obtaining or having obtained a biological sample from the subject; and
ii) performing or having performed genotyping assay on the biological sample to detect the presence of nonsense mutations in the LMOD2 gene; and
b) if a nonsense mutation is identified, administering an SBO to the subject.

2. The method of claim 1, wherein the biological sample is a whole blood sample.

3. The method of claim 1, wherein the biological sample is a saliva sample.

4. The method of claim 1, wherein the genotyping assay is sequencing.

5. The method of claim 1, wherein the SBO inhibits nonsense-mediated mRNA decay (NMD).

6. The method of claim 1, wherein the SBO prevents degradation of the LMOD2 mRNA.

7. The method of claim 1, wherein the cardiomyopathy is dilated cardiomyopathy.

8. The method of claim 1, further comprising administering one or more SBOs.

9. The method of claim 1, wherein the SBO comprises a sequence according to SEQ ID NO: 23, SEQ ID NO: 24, SEQ ID NO: 25, SEQ ID NO: 26; SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or SEQ ID NO: 41.

10. The method of claim 9, wherein the SBO is at least 95% identical to SEQ ID NO: 23, SEQ ID NO: 24, SEQ ID NO: 25, SEQ ID NO: 26; SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or SEQ ID NO: 41.

11. The method of claim 9, wherein the SBO is at least 90% identical to SEQ ID NO: 23, SEQ ID NO: 24, SEQ ID NO: 25, SEQ ID NO: 26; SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or SEQ ID NO: 41.

12. A method of treating cardiomyopathy in a subject in need thereof, said method comprising administering a steric-blocking oligonucleotide (SBO) to a subject having cardiomyopathy, to prevent degradation of leiomodin 2 (LMOD2) messenger RNA (mRNA).

13. The method of claim 12, wherein the cardiomyopathy is dilated cardiomyopathy.

14. The method of claim 12, wherein the SBO inhibits nonsense-mediated mRNA decay (NMD).

15. The method of claim 12, wherein the SBO prevents degradation of the LMOD2 mRNA.

16. The method of claim 12, further comprising administering one or more SBOs.

17. The method of claim 12, wherein the SBO comprises a sequence according to SEQ ID NO: 23, SEQ ID NO: 24, SEQ ID NO: 25, SEQ ID NO: 26; SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or SEQ ID NO: 41.

18. The method of claim 17, wherein the SBO is at least 95% identical to SEQ ID NO: 23, SEQ ID NO: 24, SEQ ID NO: 25, SEQ ID NO: 26; SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or SEQ ID NO: 41.

19. The method of claim 17, wherein the SBO is at least 90% identical to SEQ ID NO: 23, SEQ ID NO: 24, SEQ ID NO: 25, SEQ ID NO: 26; SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or SEQ ID NO: 41.

20. A steric-blocking oligonucleotide (SBO) comprising a sequence according to SEQ ID NO: 23, SEQ ID NO: 24, SEQ ID NO: 25, SEQ ID NO: 26; SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, or SEQ ID NO: 41, wherein the SBO prevents degradation of a leiomodin 2 (LMOD2) mRNA.


SEQUENCE LISTING (42 sequences)

SEQ ID NO: 1 - DNA, 35 bp, synthetic construct
  source 1..35: PCR_primers = fwd_seq: cttatgtatctcccaggcactcaccctagtcatcc, rev_seq:cctgggagatacataaggtgaagagctaggtgttcct; mol_type = other DNA
cttatgtatctcccaggcactcaccctagtcatcc

SEQ ID NO: 2 - DNA, 37 bp, synthetic construct
  source 1..37: PCR_primers = fwd_seq: cttatgtatctcccaggcactcaccctagtcatcc, rev_seq:cctgggagatacataaggtgaagagctaggtgttcct; mol_type = other DNA
cctgggagatacataaggtgaagagctaggtgttcct

SEQ ID NO: 3 - DNA, 34 bp, synthetic construct
  source 1..34: PCR_primers = fwd_seq: tccagactgtgaggagccgtcctgtctcctgtgg, rev_seq: ctcctcacagtctggacttttttggggagttttggggatg; mol_type = other DNA
tccagactgtgaggagccgtcctgtctcctgtgg

SEQ ID NO: 4 - DNA, 40 bp, synthetic construct
  source 1..40: PCR_primers = fwd_seq: tccagactgtgaggagccgtcctgtctcctgtgg, rev_seq: ctcctcacagtctggacttttttggggagttttggggatg; mol_type = other DNA
ctcctcacagtctggacttttttggggagttttggggatg

SEQ ID NO: 5 - DNA, 35 bp, synthetic construct
  source 1..35: PCR_primers = fwd_seq: cagttcctgaccttctaccccacagagatcagctc, rev_seq: gaaggtcaggaactgtcttccattttcttctcttgcac; mol_type = other DNA
cagttcctgaccttctaccccacagagatcagctc

SEQ ID NO: 6 - DNA, 38 bp, synthetic construct
  source 1..38: PCR_primers = fwd_seq: cagttcctgaccttctaccccacagagatcagctc, rev_seq: gaaggtcaggaactgtcttccattttcttctcttgcac; mol_type = other DNA
gaaggtcaggaactgtcttccattttcttctcttgcac

SEQ ID NO: 7 - DNA, 22 bp, synthetic construct
  source 1..22: PCR_primers = fwd_seq: ggacattcagcagagaggcact, rev_seq: gataagctcttcttcactttcctc; mol_type = other DNA
ggacattcagcagagaggcact

SEQ ID NO: 8 - DNA, 24 bp, synthetic construct
  source 1..24: PCR_primers = fwd_seq: ggacattcagcagagaggcact, rev_seq: gataagctcttcttcactttcctc; mol_type = other DNA
gataagctcttcttcactttcctc

SEQ ID NO: 9 - DNA, 20 bp, synthetic construct
  source 1..20: PCR_primers = fwd_seq: accccacagagatcagctca, rev_seq: tatcgcagggcttctggaac; mol_type = other DNA
accccacagagatcagctca

SEQ ID NO: 10 - DNA, 20 bp, synthetic construct
  source 1..20: PCR_primers = fwd_seq: accccacagagatcagctca, rev_seq: tatcgcagggcttctggaac; mol_type = other DNA
tatcgcagggcttctggaac

SEQ ID NO: 11 - DNA, 20 bp, synthetic construct
  source 1..20: PCR_primers = fwd_seq: acattaggagcaggcacaca, rev_seq: agggaattgggacgtgtatga; mol_type = other DNA
acattaggagcaggcacaca

SEQ ID NO: 12 - DNA, 21 bp, synthetic construct
  source 1..21: PCR_primers = fwd_seq: acattaggagcaggcacaca, rev_seq: agggaattgggacgtgtatga; mol_type = other DNA
agggaattgggacgtgtatga

SEQ ID NO: 13 - DNA, 25 bp, synthetic construct
  source 1..25: PCR_primers = fwd_seq: caccagtcagaccgatatgtcaaaa, rev_seq: tgttgtcaatgcctctgggttt; mol_type = other DNA
caccagtcagaccgatatgtcaaaa

SEQ ID NO: 14 - DNA, 22 bp, synthetic construct
  source 1..22: PCR_primers = fwd_seq: caccagtcagaccgatatgtcaaaa, rev_seq: tgttgtcaatgcctctgggttt; mol_type = other DNA
tgttgtcaatgcctctgggttt

SEQ ID NO: 15 - DNA, 20 bp, synthetic construct
  source 1..20: PCR_primers = fwd_seq: gatggattgcacgcaggttc, rev_seq: tcagagcagccgattgtctg; mol_type = other DNA
gatggattgcacgcaggttc

SEQ ID NO: 16 - DNA, 20 bp, synthetic construct
  source 1..20: PCR_primers = fwd_seq: gatggattgcacgcaggttc, rev_seq: tcagagcagccgattgtctg; mol_type = other DNA
tcagagcagccgattgtctg

SEQ ID NO: 17 - DNA, 20 bp, synthetic construct
  source 1..20: PCR_primers = fwd_seq: acagcggtcattgactggag, rev_seq: tgctgctccatacaagccaa; mol_type = other DNA
acagcggtcattgactggag

SEQ ID NO: 18 - DNA, 20 bp, synthetic construct
  source 1..20: PCR_primers = fwd_seq: acagcggtcattgactggag, rev_seq: tgctgctccatacaagccaa; mol_type = other DNA
tgctgctccatacaagccaa

SEQ ID NO: 19 - DNA, 1644 bp, Homo sapiens
  source 1..1644: mol_type = genomic DNA
atgtctacctttggctaccgaagaggactcagtaaatacgaatccatcgacgaggatgaactcctcgcctccctgtcagccgaggagctgaaggagctagagagagagttggaagacattgaacctgaccgcaaccttcccgtggggctaaggcaaaagagcctgacagagaaaacccccacagggacattcagcagagaggcactgatggcctattgggaaaaggagtcccaaaaactcttggagaaggagaggctgggggaatgtggaaaggttgcagaagacaaagaggaaagtgaagaagagcttatctttactgaaagtaacagtgaggtttctgaggaagtgtatacagaggaggaggaggaggagtcccaggaggaagaggaggaagaagacagtgacgaagaggaaagaacaattgaaactgcaaaagggattaatggaactgtaaattatgatagtgtcaattctgacaactctaagccaaagatatttaaaagtcaaatagagaacataaatttgaccaatggcagcaatgggaggaacacagagtccccagctgccattcacccttgtggaaatcctacagtgattgaggacgctttggacaagattaaaagcaatgaccctgacaccacagaagtcaatttgaacaacattgagaacatcacaacacagacccttacccgctttgctgaagccctcaaggacaacactgtggtgaagacgttcagtctggccaacacgcatgccgacgacagtgcagccatggccattgcagagatgctcaaagtcaatgagcacatcaccaacgtaaacgtcgagtccaacttcataacgggaaaggggatcctggccatcatgagagctctccagcacaacacggtgctcacggagctgcgtttccataaccagaggcacatcatgggcagccaggtggaaatggagattgtcaagctgctgaaggagaacacgacgctgctgaggctgggataccattttgaactcccaggaccaagaatgagcatgacgagcattttgacaagaaatatggataaacagaggcaaaaacgtttgcaggagcaaaaacagcaggagggatacgatggaggacccaatcttaggaccaaagtctggcaaagaggaacacctagctcttcaccttatgtatctcccaggcactcaccctggtcatccccaaaactccccaaaaaagtccagactgtgaggagccgtcctctgtctcctgtggccacacctcctcctcctccccctcctcctcctcctccccctccttcttcccaaaggctgccaccacctcctcctcctccccctcctccactcccagagaaaaagctcattaccagaaacattgcagaagtcatcaaacaacaggagagtgcccaacgggcattacaaaatggacaaaaaaagaaaaaagggaaaaaggtcaagaaacagccaaacagtattctaaaggaaataaaaaattctctgaggtcagtgcaagagaagaaaatggaagacagttcccgaccttctaccccacagagatcagctcatgagaatctcatggaagcaattcggggaagcagcataaaacagctaaagcgggtggaagttccagaagccctgcgataa

SEQ ID NO: 20 - DNA, 2855 bp, Homo sapiens
  source 1..2855: mol_type = genomic DNA
atgtctacctttggctaccgaagaggactcagtaaatacgaatccatcgacgaggatgaactcctcgcctccctgtcagccgaggagctgaaggagctagagagagagttggaagacattgaacctgaccgcaaccttcccgtggggctaaggcaaaagagcctgacagagaaaacccccacagggacattcagcagagaggcactgatggcctattgggaaaaggagtcccaaaaactcttggagaaggagaggctgggggaatgtggaaaggttgcagaagacaaagaggaaagtgaagaagagcttatctttactgaaagtaacagtgaggtttctgaggaagtgtatacagaggaggaggaggaggagtcccaggaggaagaggaggaagaagacagtgacgaagaggaaagaacaattgaaactgcaaaagggattaatggaactgtaaattatgatagtgtcaattctgacaactctaagccaaagatatttaaaagtcaaatagagaacataaatttgaccaatggcagcaatgggaggaacacagagtccccagctgccattcacccttgtggaaatcctacagtgattgaggacgctttggacaagattaaaagcaatgaccctgacaccacagaagtcaatttgaacaacattgagaacatcacaacacagacccttacccgctttgctgaagccctcaaggacaacactgtggtgaagacgttcagtctggccaacacgcatgccgacgacagtgcagccatggccattgcagagatgctcaaagtcaatgagcacatcaccaacgtaaacgtcgagtccaacttcataacgggaaaggggatcctggccatcatgagagctctccagcacaacacggtgctcacggagctgcgtttccataaccagaggcacatcatgggcagccaggtggaaatggagattgtcaagctgctgaaggagaacacgacgctgctgaggctgggataccattttgaactcccaggaccaagaatgagcatgacgagcattttgacaagaaatatggataaacagaggcaaaaacgtttgcaggagcaaaaacagcaggagggatacgatggaggacccaatcttaggaccaaagtctggcaaagaggaacacctagctcttcaccttatgtatctcccaggcactcaccctggtcatccccaaaactccccaaaaaagtccagactgtgaggagccgtcctctgtctcctgtggccacacctcctcctcctccccctcctcctcctcctccccctccttcttcccaaaggctgccaccacctcctcctcctccccctcctccactcccagagaaaaagctcattaccagaaacattgcagaagtcatcaaacaacaggagagtgcccaacgggcattacaaaatggacaaaaaaagaaaaaagggaaaaaggtcaagaaacagccaaacagtattctaaaggaaataaaaaattctctgaggtcagtgcaagagaagaaaatggaagacagttcccgaccttctaccccacagagatcagctcatgagaatctcatggaagcaattcggggaagcagcataaaacagctaaagcgggtaagtaaccagagaacagacataggggcacagataaagtaaatgagttgtcctccattgcatggtggtaccaaagtcacctctcacaatacttatcaatactttcaatattttagtatgcgagagcaaacacaccaagtttgaaacattaggagcaggcacacaagtgagcacatttctatttgagaggaacgcctgggccgctttcccagcctctcaatcatataagggcaaggaccattttcatacacgtcccaattcccttaagaagaaaaccagggaatataatctaaattccaaaaggattcaccacttgtcaaaaattttaccacagagttgtgactgattcgatacctaatttataacataaaatttataatgtggtaaaaaaattaccatgcagcaataatcaacctgagttcttctcagaggatactgctagagacatatcctacagatatttttcacttatcatgtgtgttgtttcattgagagaaaatccgctatttttgtaggtggaagttccagaagccctgcgataaaaacatgatctttagaagaggatgcagaactgttcagtggtattacatgaaatgcattgtgagatgtttctaaaataccttcttcaattcaaaatgatccctgactttaaaaataatctcacccattaattccaaagagaatcttaagaaacaatcagcatgtttcttctgtaaatatgaaaataaatttcttttttatgtcgtgagatttgtattggcaagaagcagttaatttaaagatgctcttcctatctgtggatgtgttggtaactccgagttgtaatgagttcatgaaatgtgctgttatttttgtaatctcaataaatgtggattgaagttttttccctttttttaaagccaaactaatatttttctgtgacttgatacatctgtcagatttttgtaatctcgataaatgtgtattgaagttttttccctttttttaaaaagccaaactaatatttttctgtgagttaatacatctgtcaggtgtgtatgtaacattactggacattaaaaaaaaatattacattctcacccaaaaagggtttgggtcctaagcatttgcctttctttgtttcctcttgttcaagaaaatctgattagatctctttctaaaggactgcaagcaataatttttttttatattttatttatttatttatttttttgagacaagtcttgctctgttaccca

SEQ ID NO: 21 - DNA, 3190 bp, Homo sapiens
  source 1..3190: mol_type = genomic DNA
atgtctacctttggctaccgaagaggactcagtaaatacgaatccatcgacgaggatgaactcctcgcctccctgtcagccgaggagctgaaggagctagagagagagttggaagacattgaacctgaccgcaaccttcccgtggggctaaggcaaaagagcctgacagagaaaacccccacagggacattcagcagagaggcactgatggcctattgggaaaaggagtcccaaaaactcttggagaaggagaggctgggggaatgtggaaaggttgcagaagacaaagaggaaagtgaagaagagcttatctttactgaaagtaacagtgaggtttctgaggaagtgtatacagaggaggaggaggaggagtcccaggaggaagaggaggaagaagacagtgacgaagaggaaagaacaattgaaactgcaaaagggattaatggaactgtaaattatgatagtgtcaattctgacaactctaagccaaagatatttaaaagtcaaatagagaacataaatttgaccaatggcagcaatgggaggaacacagagtccccagctgccattcacccttgtggaaatcctacagtgattgaggacgctttggacaagattaaaagcaatgaccctgacaccacagaagtcaatttgaacaacattgagaacatcacaacacagacccttacccgctttgctgaagccctcaaggacaacactgtggtgaagacgttcagtctggccaacacgcatgccgacgacagtgcagccatggccattgcagagatgctcaaagtcaatgagcacatcaccaacgtaaacgtcgagtccaacttcataacgggaaaggggatcctggccatcatgagagctctccagcacaacacggtgctcacggagctgcgtttccataaccagaggcacatcatgggcagccaggtggaaatggagattgtcaagctgctgaaggagaacacgacgctgctgaggctgggataccattttgaactcccaggaccaagaatgagcatgacgagcattttgacaagaaatatggataaacagaggcaaaaacgtttgcaggagcaaaaacagcaggagggatacgatggaggacccaatcttaggaccaaagtctggcaaagaggaacacctagctcttcaccttatgtatctcccaggcactcaccctggtcatccccaaaactccccaaaaaagtccagactgtgaggagccgtcctctgtctcctgtggccacacctcctcctcctccccctcctcctcctcctccccctccttcttcccaaaggctgccaccacctcctcctcctccccctcctccactcccagagaaaaagctcattaccagaaacattgcagaagtcatcaaacaacaggagagtgcccaacgggcattacaaaatggacaaaaaaagaaaaaagggaaaaaggtcaagaaacagccaaacagtattctaaaggaaataaaaaattctctgaggtcagtgcaagagaagaaaatggaagacagttcccgaccttctaccccacagagatcagctcatgagaatctcatggaagcaattcggggaagcagcataaaacagctaaagcgggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacaggtggaagttccagaagccctgcgataaaaacatgatctttagaagaggatgcagaactgttcagtggtattacatgaaatgcattgtgagatgtttctaaaataccttcttcaattcaaaatgatccctgactttaaaaataatctcacccattaattccaaagagaatcttaagaaacaatcagcatgtttcttctgtaaatatgaaaataaatttcttttttatgtcgtgagatttgtattggcaagaagcagttaatttaaagatgctcttcctatctgtggatgtgttggtaactccgagttgtaatgagttcatgaaatgtgctgttatttttgtaatctcaataaatgtggattgaagttttttccctttttttaaagccaaactaatatttttctgtgacttgatacatctgtcagatttttgtaatctcgataaatgtgtattgaagttttttccctttttttaaaaagccaaactaatatttttctgtgagttaatacatctgtcaggtgtgtatgtaacattactggacattaaaaaaaaatattacattctcacccaaaaagggtttgggtcctaagcatttgcctttctttgtttcctcttgttcaagaaaatctgattagatctctttctaaaggactgcaagcaataatttttttttatattttatttatttatttatttttttgagacaagtcttgctctgttaccca

SEQ ID NO: 22 - DNA, 8478 bp, Homo sapiens
  source 1..8478: mol_type = genomic DNA
atgtctacctttggctaccgaagaggactcagtaaatacgaatccatcgacgaggatgaactcctcgcctccctgtcagccgaggagctgaaggagctagagagagagttggaagacattgaacctgaccgcaaccttcccgtggggctaaggcaaaagagcctgacagagaaaacccccacagggacattcagcagagaggcactgatggcctattgggaaaaggagtcccaaaaactcttggagaaggagaggctgggggaatgtggaaaggtaggctctcgggacttttccttggctaaccccacctccccatcaccccatcccaaacccagacacttgttttccctaacttctcaatcctcatcacttgaattttcctataacccatttatacttgctactaaatcatttttcaggctgaaataatcatgtaaatgctatggccacccaatggatcataatatgtaaaattcttaattggggctctttgattgattacaaatagaaagctcaagaggaaaataattcacaatcagtgtatgggattcaatattttgccaaacataagtgttgtttgagctatatgttggtaatgtggcataatatttgttcccaagatacatgcatacgtataaatgtatacttttttttcagttaagtacatttattttctcctataaagacaaaagtgggaacgctggttgctttgactgtgatagtagctatggtgaaatagttcttttccaataccaagaagtgtggggtggtgggaagtacatgacactgagagtcacagggccttgttatgagtccctgctctgttcccacatctttagaccttgaacaatcacttttacagggttctctttccctctggacattgagggagttagagacagtgtttcttttgcgaatcaagaacccctttgagattttgataagttatgaaccttcttaagaaaatagccctgggcttgtgcaaaatacaccaacttttgcatagaatttcaaaatgataacacatcctgaaacccatctgaggactcaggggcagcacagctgtatagatgatctcttcgatttctgtctcctttacgatggtgatcctgaggttaggaaatgtgcttatatcagagctcaggcctataaagctcttgtgtactgcccagtaaacagttattttgaatttgccaaatgagtcaataccagaaggaagtccaaaagactattattctctgggaaaacagtaatgacaataaagtgtccatataacaaaaaaaaaattcttccggaattatggaagaacataggtcaggagaaacagtttaaaacccttgcactctgttggaatcatctaaaatcaaaatggcctaaagccaatggcaagacttccacagtactaatctagggctgttagatgatcttgatgatagggttatcttggccacaattgcagcaagatatgtgtgagttttgtccacgtcaatcattgcaggaagggccactgggatctggccagggtctgccaaggccacagtactaatcagctagaaggggtgtgttagaaggaaggacacatacaagtcagtacttgggcaggtcaattcctccacaaccattgttttaagagggatttttttttctttgatgtgataatgagcatcaaaataacaatgacattatcatcacaattatttaaaactctctttaataaaataaaactggctgggtatgctggctcacgcctataattcccacacttttttgggaggctgaggcaggagaatcacttgagcctaggagttcaaatccagctctggcaatatagtgagatctcatctctacaaaaaaaaaaaaaaaaaaaaaaaattagctgggtttagtggaacattagcaggtggtagtcccagctgctcaggaggctgaggtgggaggttggcttcagcatgggaggttgaggctgcagtgagctatggttgtgccactgcactcagcctgagccacaaaccaaaaccttgtctcattaaaaaaaaaaaaaaaaaaaaagaataaaactgtctcagaatatccaggctgtacaaccaatgtatcaataatccaacaaaggctttttaatgtcacaatgacatgatagtggtgggaaccaaagagcaaaacctatcgtcagccggacgaacccacctgatcctgccaaaggttttgctgatccctgccaatggcttatcagctcaattagagaggctgctgtgttgatggacttcggttcctttctaggctattaactttttctcccagtactttccctgctggccagctgtctgtagttgccaggcatgatgagcaggaacttgctgatggagggtgcaaatccaattcccacaacagggaaaatcttcgagcagctgttgggtggctgggggagcaaggtagttgcgtaagtgaagagccataaaggaaggaccctgagaaggagcggagggtcttgattgattatcaggcggctggctctgtaaaggcttataaaaagcagatgcaacacctgttgaatgccagcctttctgcccacttgggcttacacaaacttatgccgcattggggctcccagggaccctagaatcccaatggtctagctcccctattgagcagtggttaaatctcagcacaggtgccaatgagtttctcccattatctcacttcatttttaactttctgcacttgagaactgagacaaggcaaggttaagaaactcattagaggttaagagcttgtacatagaagtgaaaattaaagaacatttcaaaattgtgggtgcccggtcagcagcatcagcattacctgggatcttcttggaactgcaaattatagagttcccgtcaatctgatgaatgataaactctggggatggggcccaacaattggtgctctcacaggtgattatgacacacagtaaaacttaagaaccgctcaactacagttacctcctgaataacatgggggtcaggtgaaccaactcctgcccctgcagcatccaaaattcatgtataacttttgactcccccaaaccttaactactaatagcctactgtggacagaagccttaccaataacataaacggtcaattaacacatactttgaatgtgatatgtacatacataatagggaggtgatcaggagaaacaaccggccactcttctcattacctgatttgttttctaagtttactaacattccccttctccttgtccagtcccttgccatctcatctcccacctccttattctataccctccttcccacctccattctccaaagaatcagggttaatgccaaagcagtggcatataggaaatctgtaataaaacaggccccctagggacagagtattttataaagtaagcgaagtctgttttttgcatcctctcaccttgcctctgtagacaccttttccccttctgttctttctccagatgtcccccacctagggtgactaatcatctcagattgtccggaactatctagatttcatgactaaaatcctgggaaacccttcacactcaggcaaactgggagggtttatcacctttctcctgtgtgtagtcctgactctcagctgctgttaaccaatgtcaacttagagccattcagtaagcaaataattattgagctcctactacatgctaagcactgtgctaggtgagcaaagcagacatggtccactgctctcaacagctaacagtctgctgggaaaaacaagtgaccaaaataaagtgcgacaggtactttattgggggtgggtacaatggtatgggaatgcgtctgagggatatttggagaggatgtcaaagaaggctttctgggagatctatcatttagatgctatatgacaattcacagtttcagatattatctgtccaggtacattataaggggaaaacagatcttgcagatagagccagagggtctttaaaaaaccaaagggagtaaaatgtaatggtgtcatacaaggccgtataccttggattttgagacaggcccctttcctccaaatactgaaacagctaaaaatacatacaaacacatgaacacacacagctttacagggactagaattataaggaatttggagtggaatggggtgaggagaagtcagcccacaattctttccttcctcgggttaatccatttgtttcactgctggacagggaaggcttcctctcttccctcacgtatgcctggagacttggctagagccacactctttttcttatgttgttgcattgtgacttgcagccagctgccaccaaacatttctctctctttctctctctctctctctctccctctctctctctctctcacacacacacacacacacagagacacacagagtatctctctctctccctccctgctcctgaagaagtgaataaattaagactggaaaagtcatcccaggagtggctgaccccttccccaggccatcactcggtgcctgccactccaaacagtggataactggaggctattttgtgatagcaactgggcaaacgaacctggagacaagggcagtgttttccaaagctagatctacatgcttcaggcagcgggcgccctcgaggtcaaaaaaaaaaaagttgccaatatagagagaagaatcagggcctggaggacagggcagggtcccagggctgtagagaagataaagagaaaggactaaaaagacagcaggagaaaaagattagactctgtcagcaatttctagaccttttttttttttaatcagaaaagatagcaagaaaaagagaaacaaggggtcacttgagcgtctttgtacagcaaatcttgtaaacaggaaagacaaagatagaatgaaattgtgtttgcctgaaagcctttcataattgatacatgctaaataatttaagaccttcaggctgggcaatggcttacttcaagattttctgctgttctctgctgttccatccactttactgattagaatctagtggatgccaagggtgattatctttcccacctggtcttctacagtctgtctcatttaaccctggactagttggaggtttcaagttgattttcccaaaagggtgacctgaatgttttctctgcatcgaactggattttgacctcctgctgtcctgagcagcagccctgcctttaggcttcccttgcccagataagggacagctacagaggcagctctcaggtgattcagagagtcttctgtggtagagccctgtgccgggctctaccgtagaagcagataaataaatgctggaggagcaaatgacagctctgcttctgtctacactgaggcaaggcctggcagagagtggttggcttctgtcacatctgctatggatggaaaatttgggaatttttgaattaaaaagggaaactcattaacatattgtatatttttgttgtcatttgattgcttgagatggagaaataagaaagaacagagtgattccagaagggagggttgaatttgctacattgtaagtaaatggagagtaaaccagtaagagtcggtcctcaggaggtgaacaaatttatcaagggttttccaaagctgaagttctgatttatttgttcaaattagcaaaatgattatatatttttattcaataagttgaattctggatattctgcagaaaacaagagaaatgtttgtagaaaaagctattgctatttattgcaataaaatgtcagccttggtcatgagaactagacagaatggaagcagaggattattaactttttttattataaacttatgtcacttgccatgtgctacttttgcaagttaaaaaatgtacttaaaaatggtatcatttttaagaagcttaatgatgatatcatactctttaggttgcagaagacaaagaggaaagtgaagaagagcttatctttactgaaagtaacagtgaggtttctgaggaagtgtatacagaggaggaggaggaggagtcccaggaggaagaggaggaagaagacagtgacgaagaggaaagaacaattgaaactgcaaaagggattaatggaactgtaaattatgatagtgtcaattctgacaactctaagccaaagatatttaaaagtcaaatagagaacataaatttgaccaatggcagcaatgggaggaacacagagtccccagctgccattcacccttgtggaaatcctacagtgattgaggacgctttggacaagattaaaagcaatgaccctgacaccacagaagtcaatttgaacaacattgagaacatcacaacacagacccttacccgctttgctgaagccctcaaggacaacactgtggtgaagacgttcagtctggccaacacgcatgccgacgacagtgcagccatggccattgcagagatgctcaaagtcaatgagcacatcaccaacgtaaacgtcgagtccaacttcataacgggaaaggggatcctggccatcatgagagctctccagcacaacacggtgctcacggagctgcgtttccataaccagaggcacatcatgggcagccaggtggaaatggagattgtcaagctgctgaaggagaacacgacgctgctgaggctgggataccattttgaactcccaggaccaagaatgagcatgacgagcattttgacaagaaatatggataaacagaggcaaaaacgtttgcaggagcaaaaacagcaggagggatacgatggaggacccaatcttaggaccaaagtctggcaaagaggaacacctagctcttcaccttatgtatctcccaggcactcaccctggtcatccccaaaactccccaaaaaagtccagactgtgaggagccgtcctctgtctcctgtggccacacctcctcctcctccccctcctcctcctcctccccctccttcttcccaaaggctgccaccacctcctcctcctccccctcctccactcccagagaaaaagctcattaccagaaacattgcagaagtcatcaaacaacaggagagtgcccaacgggcattacaaaatggacaaaaaaagaaaaaagggaaaaaggtcaagaaacagccaaacagtattctaaaggaaataaaaaattctctgaggtcagtgcaagagaagaaaatggaagacagttcccgaccttctaccccacagagatcagctcatgagaatctcatggaagcaattcggggaagcagcataaaacagctaaagcgggtaagtaaccagagaacagacataggggcacagataaagtaaatgagttgtcctccattgcatggtggtaccaaagtcacctctcacaatacttatcaatactttcaatattttagtatgcgagagcaaacacaccaagtttgaaacattaggagcaggcacacaagtgagcacatttctatttgagaggaacgcctgggccgctttcccagcctctcaatcatataagggcaaggaccattttcatacacgtcccaattcccttaagaagaaaaccagggaatataatctaaattccaaaaggattcaccacttgtcaaaaattttaccacagagttgtgactgattcgatacctaatttataacataaaatttataatgtggtaaaaaaattaccatgcagcaataatcaacctgagttcttctcagaggatactgctagagacatatcctacagatatttttcacttatcatgtgtgttgtttcattgagagaaaatccgctatttttgtaggtggaagttccagaagccctgcgataaaaacatgatctttagaagaggatgcagaactgttcagtggtattacatgaaatgcattgtgagatgtttctaaaataccttcttcaattcaaaatgatccctgactttaaaaataatctcacccattaattccaaagagaatcttaagaaacaatcagcatgtttcttctgtaaatatgaaaataaatttcttttttatgtcgtgagatttgtattggcaagaagcagttaatttaaagatgctcttcctatctgtggatgtgttggtaactccgagttgtaatgagttcatgaaatgtgctgttatttttgtaatctcaataaatgtggattgaagttttttccctttttttaaagccaaactaatatttttctgtgacttgatacatctgtcagatttttgtaatctcgataaatgtgtattgaagttttttccctttttttaaaaagccaaactaatatttttctgtgagttaatacatctgtcaggtgtgtatgtaacattactggacattaaaaaaaaatattacattctcacccaaaaagggtttgggtcctaagcatttgcctttctttgtttcctcttgttcaagaaaatctgattagatctctttctaaaggactgcaagcaataatttttttttatattttatttatttatttatttttttgagacaagtcttgctctgttaccca

SEQ ID NO: 23 - DNA, 21 bp, synthetic construct
  source 1..21: mol_type = other DNA
gctttagctgttttatgctgc

SEQ ID NO: 24 - DNA, 22 bp, synthetic construct
  source 1..22: mol_type = other DNA
gctttagctgttttatgctgct

SEQ ID NO: 25 - DNA, 22 bp, synthetic construct
  source 1..22: mol_type = other DNA
ctttagctgttttatgctgctt

SEQ ID NO: 26 - DNA, 22 bp, synthetic construct
  source 1..22: mol_type = other DNA
tttagctgttttatgctgcttc

SEQ ID NO: 27 - DNA, 20 bp, synthetic construct
  source 1..20: mol_type = other DNA
agctgttttatgctgcttcc

SEQ ID NO: 28 - DNA, 20 bp, synthetic construct
  source 1..20: mol_type = other DNA
gctgttttatgctgcttccc

SEQ ID NO: 29 - DNA, 20 bp, synthetic construct
  source 1..20: mol_type = other DNA
ctgttttatgctgcttcccc

SEQ ID NO: 30 - DNA, 20 bp, synthetic construct
  source 1..20: mol_type = other DNA
tgttttatgctgcttccccg

SEQ ID NO: 31 - DNA, 20 bp, synthetic construct
  source 1..20: mol_type = other DNA
gttttatgctgcttccccga

SEQ ID NO: 32 - DNA, 20 bp, synthetic construct
  source 1..20: mol_type = other DNA
ttttatgctgcttccccgaa

SEQ ID NO: 33 - DNA, 20 bp, synthetic construct
  source 1..20: mol_type = other DNA
tttatgctgcttccccgaat

SEQ ID NO: 34 - DNA, 20 bp, synthetic construct
  source 1..20: mol_type = other DNA
ttatgctgcttccccgaatt

SEQ ID NO: 35 - DNA, 20 bp, synthetic construct
  source 1..20: mol_type = other DNA
tatgctgcttccccgaattg

SEQ ID NO: 36 - DNA, 20 bp, synthetic construct
  source 1..20: mol_type = other DNA
atgctgcttccccgaattgc

SEQ ID NO: 37 - DNA, 20 bp, synthetic construct
  source 1..20: mol_type = other DNA
tgctgcttccccgaattgct

SEQ ID NO: 38 - DNA, 20 bp, synthetic construct
  source 1..20: mol_type = other DNA
gctgcttccccgaattgctt

SEQ ID NO: 39 - DNA, 20 bp, synthetic construct
  source 1..20: mol_type = other DNA
ctgcttccccgaattgcttc

SEQ ID NO: 40 - DNA, 20 bp, synthetic construct
  source 1..20: mol_type = other DNA
tgcttccccgaattgcttcc

SEQ ID NO: 41 - DNA, 20 bp, synthetic construct
  source 1..20: mol_type = other DNA
gcttccccgaattgcttcca

SEQ ID NO: 42 - DNA, 20 bp, synthetic construct
  source 1..20: mol_type = other DNA
tctaactacgccttggtttc